(19) United States
(12) Patent Application Publication
(10) Pub. No.: US 2025/0244340 A1
(43) Pub. Date: Jul. 31, 2025

(54) DIAGNOSING AND TREATING A PATHOLOGICAL CONDITION OF THE INTESTINAL TRACT

(71) Applicant: MAGNOSTICS LIMITED, Monkstown (IE)

(72) Inventors: Peng LI, Monkstown (IE); Gil LEE, Monkstown (IE); Anne CHEVALIER, Monkstown (IE)

(21) Appl. No.: 18/878,737

(22) PCT Filed: Jun. 24, 2023
(86) PCT No.: PCT/IB2023/056537

§ 371 Date: Dec. 24, 2024

Related U.S. Application Data
(60) Provisional application No. 63/355,590, filed on Jun. 25, 2022.

(51) Int. Cl. G01N 33/68; G01N 33/543

(52) U.S. Cl. CPC G01N 33/6893; G01N 33/54326; G01N 2333/4734; G01N 2333/91177; G01N 2800/06

(57) ABSTRACT

The present disclosure contemplates detecting and treating a pathological condition, such as intestinal ischemia such as acute mesenteric ischemia, necrotizing enterocolitis, inflammatory bowel disease, and bowel graft rejection in a subject's intestinal tract. The methodology comprises obtaining a sample from the subject; detecting whether an analyte such as Villin-1, α-glutathione S-transferase, or intestinal fatty acid binding protein (I-FABP) is present in the sample by contacting the sample with an anti-analyte antibody and detecting binding between analyte and the antibody; and diagnosing the subject with the condition when, for example, the presence of analyte in the sample is detected and exceeds the level of analyte in a healthy control sample. A superparamagnetic bead includes an anti-Villin-1 antibody; an anti-α-glutathione S-transferase antibody, or an anti-intestinal-fatty acid binding protein (I-FABP) antibody. A superparamagnetic bead comprising a surface coating that binds Villin-1, α-glutathione S-transferase, or intestinal-fatty acid binding protein (I-FABP).

### CROSS REFERENCE

The present application claims priority benefit of U.S. Provisional Application No. 63/355,590, filed 25 Jun. 2022, the entire contents of which application are hereby incorporated herein by reference.

### FIELD OF USE

The present application relates to methodology for detecting and treating a pathological condition, such as intestinal ischemia, in a subject's intestinal tract.

### INTRODUCTION

Abdominal pain comprises 5 to 10 percent of emergency department visits and the diagnosis of the underlying causes remains extremely challenging despite the available diagnostic tests (1-5). Undifferentiated abdominal pain remains the diagnosis for approximately 25 percent of patients discharged from the emergency department and between 35 and 41 percent for those admitted to the hospital (6-8). Delayed diagnosis of the underlying causes of abdominal pain results in increased mortality, especially in the elderly (9, 10). The possible underlying causes of abdominal pain ranges from causes that disappear spontaneously to potentially lethal causes such as intestinal ischemia (8). See FIG. 1, Diagnosis and Management of Acute Bowel Ischemia. From ACS Surgery: Principles and Practices.

Intestinal ischemia continues to be a catastrophic emergency with an estimated prevalence of up to 1 in 1000 hospital admissions (11, 12). In hospital, mortality of acute mesenteric ischemia (AMI) ranges between 60-80% and hasn't changed in the last decades (13-15). Necrosis of the bowel results in bacterial translocation leading to in sepsis and death (16, 17). Also, following restauration of blood flow to the ischemic intestine, injury to the intestinal tissues continues as reperfusion is associated with oxidative stress and activation of the innate immune system. This phenomenon is known as ischemia-reperfusion injury (IRI). The diagnosis of intestinal ischemia is notoriously challenging and time-consuming, as symptoms can be subtle and clinical examination often does not reflect the degree of infarction. To improve outcome, an early diagnosis is essential and, and despite a set of radiologic evaluation tools, timely diagnosis depends on an easily accessible, accurate and specific marker (18, 19).

Thus, the primordial objective in patients with abdominal pain is to exclude an acute mesenteric ischemia and this triage is currently done by pre-test probability assessment, computed tomography scan (CT scan) and nonspecific biomarker measurements. A point-of-care screenings test for intestinal ischemia could prevent unnecessary referral to emergency departments by general physicians and avoid the costs associated with diagnostic and therapeutic work up of abdominal pain at the emergency departments. Currently, however, there is no established serologic marker available that allows for accurate screening for intestinal ischemia.

In the last decade, several markers have been proposed as promising e.g., glutathione-S-transferase, citrulline, or intestinal-fatty acid binding protein (I-FABP) (20). However, none of these has shown superior performance and non-specific markers like lactate, lactate dehydrogenase or D-dimers are still used in daily clinical practice (21, 22). Therefore, a plasma marker that is undetectable in a healthy physiological situation, that is relatively specific to the intestine, sensitive to early ischemia and is able to guide the clinician through the process of perfusion is urgently needed.

Villin-1 was first described as a major microfilament-associated protein—95 kDa—of the intestinal microvillus (23). It belongs to the Gelsolin family of calcium-regulated actin-binding proteins (24, 25). In vitro and in animal experiments, Villin-1 synthesis increases during enterocyte differentiation (26). Therefore, Villin-1 expression is highest in the more mature enterocytes, located at the tip of the villi. Interestingly these mature enterocytes at the villus tip are most sensitive to IRI. In the clinical setting, Villin-1 has been described as a marker for colon cancer and micrometastasis (27-30).

### SUMMARY

As disclosed herein, diagnosing a pathological condition, such as ischemia (including acute mesenteric ischemia), necrotizing enterocolitis, inflammatory bowel disease, and bowel graft rejection in a subject's intestinal tract, has been facilitated by detecting an analyte such as Villin-1, α-glutathione S-transferase, or intestinal-fatty acid binding protein (I-FABP), in samples from a subject, which makes it possible for earlier diagnosis and subsequent treatment of the pathological condition. Although detectable in other ways, in some embodiments, a superparamagnetic bead comprising an anti-Villin-1 antibody or an anti-α-glutathione S-transferase antibody, or an anti-intestinal-fatty acid binding protein antibody make it possible to rapidly extract analyte from the sample, which makes it possible for earlier diagnosis and subsequent treatment initiation or modification.

A method detects and treats a pathological condition in an intestinal tract of a subject. The method includes contacting a biological sample from the subject with an analyte receptor and detecting binding between the analyte receptor and the analyte when the analyte is present in the biological sample. The method includes diagnosing the subject with the pathological condition. The analyte in the sample is detected and is present in an amount that exceeds a predetermined level. The analyte has a receptor and is chosen from Villin-1, α-glutathione S-transferase, and I-FABP.

In some embodiments, the pathological condition is selected from intestinal ischemia (acute mesenteric ischemia), necrotizing enterocolitis, inflammatory bowel disease, and bowel graft rejection. In some embodiments, the pathological condition is intestinal ischemia, such as acute mesenteric ischemia, and optionally, after diagnosing the subject with intestinal ischemia such as acute mesenteric ischemia, performing a contrast angiography to confirm the diagnosis and/or to detect a blockage or defect in an affected blood vessel, such as an artery, vein, or capillary. When used with contrast angiography, the time to treatment is vastly improved and the outcome is improved.

In some embodiments, the detecting binding between the analyte and the analyte receptor chosen from: a Villin-1 and an anti-Villin-1 antibody performed by an immunoassay, such as a lateral flow assay; a α-glutathione S-transferase and an anti-α-glutathione S-transferase antibody performed by an immunoassay, such as a lateral flow assay; and an intestinal-fatty acid binding protein (I-FABP) and an anti-I-FABP antibody performed by an immunoassay, such as a lateral flow assay.

In some embodiments, after diagnosing the subject with the pathological condition, the method further comprises, administering an effective amount of an anticoagulant, antibiotic, or both, to the subject or performing surgery to treat the pathological condition.

In some embodiments, the subject has one or more symptoms chosen from abdominal pain; an urgent need to have a bowel movement; frequent, forceful bowel movements; abdominal tenderness or distention; blood in the subject's stool; and mental confusion. In some embodiments, the subject has abdominal pain.

In some embodiments, the method thereafter includes initiating or changing treatment of intestinal ischemia (including acute mesenteric ischemia), including administering to the diagnosed subject an effective amount of one or more medicines for the treatment of intestinal ischemia or performing surgery to treat the pathological condition, especially acute mesenteric ischemia. In some embodiments, the one or more medicines comprise one or more anticoagulants. In some embodiments, the anticoagulants are chosen from warfarin, heparin, rivaroxaban (Xarelto) dabigatran (Pradaxa) apixaban (Eliquis), and edoxaban (Lixiana). In some embodiments, the one or more medicines comprises antibiotics, such as Penicillins, such as penicillin V potassium, amoxicillin, amoxicillin/clavulanate (Augmentin); Tetracyclines, such as doxycycline, tetracycline, and minocycline; Cephalosporins, such as cefuroxime (Ceftin), ceftriaxone (Rocephin), and Cefdinir (Omnicef); Quinolones, such as ciprofloxacin (Cipro), levofloxacin (Levaquin), and moxifloxacin (Avelox); Lincomycins, such as clindamycin (Cleocin) and lincomycin (Lincocin); Macrolides, such as azithromycin (Zithromax), clarithromycin (Biaxin), and erythromycin; Sulfonamides, such as sulfamethoxazole-trimethoprim (Bactrim, Bactrim DS, Septra), sulfasalazine (Azulfidine), and sulfisoxazole (optionally combined with erythromycin); Glycopeptides, such as dalbavancin (Dalvance), oritavancin (Orbactiv), telavancin (Vibativ), and vancomycin (Vancocin); Aminoglycosides, such as gentamicin, tobramycin, and amikacin; and Carbapenems, such as imipenem/cilastatin (Primaxin), meropenem (Merrem), doripenem (Doribax), and ertapenem (Inanz). In some embodiments, the antibiotics are chosen from aminoglycosides, ampicillin, amoxicillin, amoxicillin/clavulanic acid (Augmentin), carbapenems (e.g., imipenem), piperacillin/tazobactam, quinolones (e.g., ciprofloxacin), tetracyclines, chloramphenicol, ticarcillin, trimethoprim/sulfamethoxazole (Bactrim), doxycycline, cephalexin, clindamycin, metronidazole, azithromycin, and levofloxacin. In some embodiments, the antibiotics are chosen from broad spectrum antibiotics.

In some embodiments, the condition is necrotizing enterocolitis.

In some embodiments, the subject has one or more symptoms chosen from abdominal distention (bloating or swelling); feedings stay in the stomach instead of moving through to the intestines as normal; bile-colored (greenish) fluid in the stomach; bloody bowel movements; and signs of infection such as apnea (stopping breathing), low heart rate, lethargy (sluggishness).

In some embodiments, the method thereafter includes initiating or changing treatment of necrotizing enterocolitis, comprising administering to the diagnosed subject an effective amount of one or more medicines for the treatment of necrotizing enterocolitis. In some embodiments, the one or more medicines comprises antibiotics as noted herein.

In some embodiments, the condition is inflammatory bowel disease, such as ulcerative colitis or Crohn's disease.

In some embodiments, the subject has one or more symptoms chosen from diarrhea, abdominal pain, fatigue and weight loss.

In some embodiments, the method thereafter includes initiating or changing treatment of inflammatory bowel disease, comprising administering to the diagnosed subject an effective amount of the one or more medicines for the treatment of inflammatory bowel disease. In some embodiments, the one or more medicines are chosen from antibiotics, anti-inflammatory drugs, and immune system suppressors. In some embodiments, the one or more medicines comprises antibiotics as noted herein. In some embodiments, the one or more medicines comprises anti-inflammatory drugs, such as corticosteroids and aminosalicylates. In some embodiments, the anti-inflammatory drugs are chosen from mesalamine (such as Asacol HD, Delzicol, and others), balsalazide (Colazal) and olsalazine (Dipentum). In some embodiments, the one or more medicines comprises immune system suppressors, such as TNF-alpha inhibitors and biologics. In some embodiments, the immune system suppressors are chosen from infliximab (Remicade), adalimumab (Humira), golimumab (Simponi), natalizumab (Tysabri), vedolizumab (Entyvio), and ustekinumab (Stelara).

In some embodiments, the condition is bowel graft rejection. In some embodiments, the subject has no symptoms or one or more symptoms chosen from fever, malaise, change in ostomy output (increased or decreased), intestinal bleeding, nausea, and vomiting. In some embodiments, the method thereafter includes changing treatment of bowel graft rejection, comprising administering to the diagnosed subject an effective amount of one or more medicines for the treatment of bowel graft rejection different than earlier treatments. In some embodiments, the one or more medicines are chosen from antibiotics and anti-rejection medicines. In some embodiments, the one or more medicines comprises antibiotics. In some embodiments, the antibiotics are chosen from those noted herein. In some embodiments, the one or more medicines include anti-rejection medicines, such as immunosuppressive agents like Tacrolimus (Prograf), Sirolimus (Rapamune), Steroids, and Everaloums.

In some embodiments, the subject is chosen from humans (including infants) and other mammal animals, such as pets (dogs, cats, etc.), livestock (cattle, pigs, goats, sheep, horses, mules, donkeys, rabbits, etc.).

In some embodiments, the biological sample is serum.

In some embodiments, the analyte receptor is an anti-Villin-1 antibody that is C-terminus or N-terminus type; an anti-α-glutathione S-transferase antibody that is C-terminus or N-terminus type; or an anti-intestinal-fatty acid binding protein (I-FABP) antibody C-terminus or N-terminus type.

In some embodiments, the analyte receptor is an anti-Villin-1 antibody that is polyclonal or monoclonal, an anti-α-glutathione S-transferase antibody that is polyclonal or monoclonal, or an anti-intestinal-fatty acid binding protein (I-FABP) antibody that is polyclonal or monoclonal.

In some embodiments, the analyte receptor is a Villin-1 receptor chosen from an anti-Villin-1 antibody, which is chosen from SP145 from, e.g., Invitrogen, UMAB230 from, e.g., OriGene, OTI3B3 from OriGene 3E5G11 from, e.g., Abcam, EPR3490 from, e.g., Abcam, VIL1 from, e.g., Abbexa, AS1A11 from, e.g., G Biosciences, VIL1/1314 from, e.g., enquire BioReagents, 1D2C3 from, e.g., Santa Cruz Biotechnology, OAGA00811 from, e.g., Aviva Systems Biology, OAEB02383 from, e.g., Aviva Systems Biology, and R814 from, e.g., Cell Signaling Technology; or a α-glutathione S-transferase receptor chosen from an anti-α-glutathione S-transferase antibody, which is chosen from: e.g., anti-α-glutathione S-transferase antibody, Merck, e.g., GSTA1/α-glutathione S-transferase antibody, BioOrbyt, e.g., anti-α-glutathione S-transferase antibody, Sigma, e.g., anti-α-glutathione S-transferase antibody, Abbexa, e.g., Glutathione S Transferase alpha 1 (GSTA1) Rabbit Polyclonal Antibody, Origene, e.g., GSTA1 Polyclonal Antibody, ThermoFisher Scientific, e.g., Glutathione S-Transferase alpha 3, Antibodies-online.com, e.g., Glutathione S-Transferase alpha, Cloud-Clone Corp, e.g., Glutathione S-transferase alpha, Boster Bio, e.g., Glutathione S-transferase alpha, Bio-Techne, e.g., Glutathione S-transferase alpha, Absolute Antibody, e.g., Glutathione S-transferase alpha, Cambridge Bioscience; or an intestinal-fatty acid binding protein (I-FABP) receptor chosen from an anti-I-FABP antibody, which is chosen from: e.g., Mouse anti-Human I-FABP/FABP2 Monoclonal Antibody (MBS246348), MyBioSource.com, e.g., Monoclonal Mouse anti-Human I-FABP/FABP2 Antibody, Lifespan Biosciences, e.g., anti-I-FABP antibody: Rabbit anti-Human I-FABP Polyclonal Antibody, MyBioSource.com, e.g., Mouse anti-Human FABP2 Monoclonal Antibody, ProteinTech, e.g., Fatty Acid Binding Protein 2, Intestinal (FABP2) Polyclonal Antibody, Biomatik, e.g., Recombinant Anti-I-FABP antibody, abCam, e.g., Rabbit Anti-Human FABP2/I-FABP pAb, Cell Sciences, e.g., Rat FABP2/I-FABP Biotinylated Antibody, R&D Systems, e.g., Rabbit Anti-Human FABP, Biorbyt, e.g., FABP2/I-FABP Antibody, Novus Biologicals, e.g., Intestinal Fatty Acid Binding Protein/I-FABP (FABP2) Antibody, Abbexa Ltd, e.g., Anti-FABP2 antibody, St. John's Laboratory, e.g., Anti-FABP2/I-FABP Antibody, BosterBio, e.g., Anti-FABP2, GeneTex, e.g., Anti-FABP2 Antibody, Rabbit Polyclonal, SionBiological, e.g., FABP2 antibody (Fatty Acid Binding Protein 2, Intestinal), Antibodies online, e.g., Rabbit Anti-FABP2, US Biological, e.g., FABP2 Polyclonal Antibody, Elabscience, e.g., I-FABP Polyclonal Antibody, G-Biosciences, e.g., I-FABP Antibody, Santa Cruz Biotechnology, Inc., e.g., I-FABP Antibody, Hycult Biotech, e.g., FABP2 Antibody, Thermo Fisher Scientific, e.g., FABP2 Antibody, NSJ Bioreagents, e.g., FABP2 Antibody, RayBiotech; e.g., FABP2 Antibody, AssayPro, e.g., FABP2 Antibody, Affinity Biosciences, e.g., FABP2 Antibody, OriGene Technologies, e.g., FABP2 Antibody, Cayman Chemical, e.g., FABP2 Antibody, Proteintech Group Inc, or e.g., FABP2 Antibody, ProSci.

In some embodiments, detecting binding is between Villin-1 and the antibody; α-glutathione S-transferase and the antibody; or intestinal-fatty acid binding protein (I-FABP) and the antibody, and detecting binding lasts a period of time less than 120 minutes or 60 minutes or 30 minutes.

In some embodiments, detecting binding between Villin-1 and the antibody is performed by an immunological assay, such as a lateral flow assay; or detecting binding between α-glutathione S-transferase and the antibody is performed by an immunological assay, such as a lateral flow assay; or detecting binding between I-FABP and the antibody is performed by an immunological assay, such as a lateral flow assay. In some embodiments, the immunological assay is chosen from ELISA. In some embodiments, the immunological assay is a lateral flow assay. In some embodiments, detecting binding between Villin-1 and the antibody is performed using a superparamagnetic bead comprising an anti-Villin-1 antibody; or detecting binding between α-glutathione S-transferase and the antibody is performed using a superparamagnetic bead comprising an anti-α-glutathione S-transferase antibody; or detecting binding between I-FABP and the antibody is performed using a superparamagnetic bead comprising an anti-I-FABP antibody.

In some embodiments, the predetermined level is a normal amount of analyte in a healthy control sample or a measured amount of analyte from a biological sample taken from the same subject at an earlier time. In some embodiments, the predetermined level is a normal amount of analyte in a healthy control sample or a measured amount of analyte from a biological sample taken from the same subject at an earlier time or a level from defined severity to the severity to the intestinal tissue.

A superparamagnetic bead comprises an anti-Villin-1 antibody; an anti-α-glutathione S-transferase antibody, or an anti-intestinal-fatty acid binding protein (I-FABP) antibody.

A superparamagnetic bead comprises a surface coating that binds Villin-1, α-glutathione S-transferase, or intestinal-fatty acid binding protein (I-FABP). In some embodiments, the surface coating comprises a receptor or aptamer.

A kit includes superparamagnetic beads comprising an anti-Villin-1 antibody, anti-α-glutathione S-transferase antibody, or anti-intestinal-fatty acid binding protein (I-FABP) antibody.

Use of any described substance or composition or kit for diagnosing a pathological condition, such as ischemia (including acute mesenteric ischemia), necrotizing enterocolitis, inflammatory bowel disease, and bowel graft rejection in a subject's intestinal tract, in any method described herein.

Additional objects and advantages of the invention will be set forth in part in the description which follows, and in part will be obvious from the description, or may be learned by practice of the embodiments disclosed herein. The objects and advantages will be realized and attained by means of the elements and combinations particularly pointed out in the appended claims.

It is to be understood that both the foregoing general description and the following detailed description are exemplary and explanatory only and are not necessarily restrictive of the subject matter as claimed.

The accompanying drawings, which are incorporated in and constitute a part of this specification, illustrate embodiments and together with the description, serve to explain principles.

### BRIEF DESCRIPTION OF THE DRAWINGS

FIG. 1. Workflow for the diagnosis management of acute bowel ischemia including acute mesenteric ischemia.

FIG. 2. Schematic of the direct (A) and indirect (B) assay configurations that may be used to detect Villin-1. (1) substrate used to bind Villin-1 from clinical sample; (2) linker used to immobilize receptor on substrate; (3) receptor used to capture Villin-1; (4) Villin-1, (5) receptor used to detect villin-1; (6) linker used to immobilize receptor on substrate; (7) detector used to detect Villin-1.

FIG. 3. Steps that are used to detect Villin-1 in different analysis formats. A. Detection using two receptor reactions and two rinsing steps in a protocol that would be typical of automated in vitro diagnostics used in clinical laboratories. B. Detection with a single reaction step that requires receptors that do not cross react in serum and an analyzer that does not require separation. C. Lateral flow assay format.

FIG. 4. Villin-1 ELISA results demonstrating that Villin-1 was detectable to a sensitivity of 5 ng/mL with antibodies directed against both the C and N-terminus component of the protein, i.e., the antibodies were polyclonal R814 and monoclonal 3E5G11. The background for both the anti-mouse-HRP and anti-rabbit-HRP assays was 0.045±0.05.

FIG. 5. Villin-1 sandwich ELISA results demonstrating that Villin-1 was detectable to a sensitivity of 5 ng/mL. The polyclonal R814 antibody was adsorbed to the plate and the monoclonal 3E5G11 antibody against the C-terminal domain was used for detection. The background measured for anti-mouse-HRP assays was 0.045±0.05.

FIG. 6. Villin-1 ELISA results in serum demonstrating that Villin-1 was detectable to a sensitivity of 5 ng/mL in 10% and 25% serum. The sensitivity decreased to 20 ng/mL in 50% serum. The background measured for anti-rabbit-HRP assays in 10%, 25% and 50% serum were 0.05±0.05, 0.06±0.05 and 0.10±0.05, respectively.

FIG. 7. Detection of Villin-1 using superparamagnetic beads assay in which streptavidin functionalized superparamagnetic beads are coated with biotinylated monoclonal 3E5G11 antibody. Villin-1 was detectable to a sensitivity of 5 ng/ml in 10% and 25% serum. The sensitivity decreased to 20 ng/mL in 50% serum. The time of the assay was decreased to approximately 30 minutes.

FIG. 8. Picture showing the results of a half-strip lateral flow assay to detect Villin-1 in buffer. Each prototype was prepared in half-strip format, containing an absorbent pad and a detection pad. Prior to assembly, the detection pad was stripped with the capture polyclonal R814 antibody on the test line and with the anti-mouse-HRP control antibody on the control line. Gold nanoparticles were conjugated with the detection mouse monoclonal 3E5G11 antibody. The assay was run by incubating the samples (in buffer) and the conjugated nanoparticles for 30 minutes at 37° C., followed by addition of the half-strips into the solution. Following addition of a running buffer to ensure complete migration, the results were evaluated by naked eye. All tests were showing a red dot at the control line (highlighting the validity of the assay). A dot was visible at the test line for samples containing at least 50 ng/ml of Villin-1 (with a color gradient corresponding to the increase in the levels of Villin-1). A very light signal was visible for the sample containing 10 ng/ml of Villin-1.

FIG. 9. Picture showing the results of a half-strip lateral flow assay to detect Villin-1 in 50% serum. Each prototype was prepared in half-strip format, containing an absorbent pad and a detection pad. Prior to assembly, the detection pad was stripped with the capture polyclonal R814 antibody on the test line and with the anti-mouse-HRP control antibody on the control line. Gold nanoparticles were conjugated with the detection mouse monoclonal 3E5G11 antibody. The assay was run by incubating the samples (in 50% serum) and the conjugated nanoparticles for 30 minutes at 37° C., followed by addition of the half-strips into the solution. Following addition of a running buffer to ensure complete migration, the results were evaluated by naked eye. All tests showed a red dot at the control line (highlighting the validity of the assay). A dot was visible at the test line for samples containing at least 500 ng/mL of Villin-1 (with a color gradient corresponding to the increase in the levels of Villin-1). A very light signal was visible for the sample containing 100 ng/ml.

FIG. 10. Table 1. Villin-1 superparamagnetic bead assay. Fluorescence values for the sandwich ELISA on superparamagnetic beads for the concentrations of Villin-1 performed on different animal model samples.

### DETAILED DESCRIPTION

Reference will now be made in detail to embodiments, examples of which are illustrated in the accompanying drawings. Wherever possible, the same reference numbers will be used throughout the drawings to refer to the same or like parts.

Recently, it was shown in a rat model of intestinal ischemia that Villin-1 is a promising serological marker for intestinal IRI, that correlated stronger than I-FABP to histologic alterations and permeability. Also, in humans, the release of Villin-1 was detected in the plasma of subjects subjected to controlled intestinal IRI. Based on these findings, Villin-1 could become a valuable biomarker for the early diagnosis of a pathological intestinal condition, such as intestinal ischemia, and for guidance of the clinicians through the process of reperfusion.

The development of a point-of-care test for the detection of Villin-1 in samples could provide an important step forward in the management of subjects with abdominal pain, both in the ambulatory care and hospital setting.

As explained below, each present inventor developed methodology for detecting a pathological condition, such as intestinal ischemia (including acute mesenteric ischemia), necrotizing enterocolitis, inflammatory bowel disease, and bowel graft rejection, in a subject's intestinal tract, facilitated by detecting Villin-1 in a sample from the subject. Such methodology permits earlier diagnosis and subsequent treatment of the pathological condition. Although detectable in other ways, in some embodiments, magnetic bead assays make it possible to rapidly extract Villin-1 from the sample, thereby providing earlier diagnosis and subsequent treatment initiation or modification.

The methodology permits: (1) the assessment of the levels of Villin-1 in biological samples, and comparison to a reference level representing the threshold to determine if a subject has or does not have the pathological condition; (2) the assessment of the levels of Villin-1 in biological samples, and comparison to reference levels associated with preselected severity to predict the levels of damages to the intestinal tissues of the subject; or (3) the assessment of the levels of Villin-1 in biological samples collected from a subject during and after therapies, and comparison to levels measured in prior samples, wherein a difference in Villin-1 levels would be correlated with a change in subject's condition.

In the third case, an increase in the levels of Villin-1 relative to the levels measured in first sample would be indicative of a need for treatment adjustment, which could include surgery due to additional damages and necrosis to the intestinal tissues of the subject.

In cases (1)-(3), the pathological condition is the obstruction of a blood vessel leading to hypoperfusion of the intestinal tissues, including mesenteric arterial embolism, mesenteric arterial thrombosis, mesenteric venous thrombosis and nonocclusive mesenteric ischemia.

In any of the cases, the sample is blood, serum or plasma, collected from the subject healthy, sick or in recovery.

In any of the above cases, the levels of Villin-1 in samples are determined through a variety of biochemical, immunological, molecular methods including but not limited to ELISA, superparamagnetic beads assay, lateral flow assays, and other kits comprising at least one antibody that binds specifically to Villin-1.

In any of the above cases, at least 1 other biomarker can be measured in a panel including Villin-1 (selected from the group: I-FABP, D-dimer, D-lactate, citrulline, and the like).

In any of the above cases, the methods that can be used alone or in combination with other medical/clinical interventions to help diagnosis and prognosis of acute mesenteric ischemia (AMI) in subjects, as deemed appropriate by those who know the art.

In any of the above cases, the methods can involve or not the comparison to at least one reference control, which can be either a negative control, a positive control or a various control containing known concentration of Villin-1 (or an analog) (for realization of a standard curve).

Technical terminology in this description conforms to common usage in the art of biomarkers, and representative protocols may be found in, for example, Current Protocols in Molecular Biology (Ausubel, F. M. et al., John Wiley and Sons, Inc., Media Pa.).

As used herein, the term “subject” refers to a vertebrate suspected of having a pathological condition of the intestinal track, such as intestinal ischemia (including acute mesenteric ischemia), necrotizing enterocolitis, inflammatory bowel disease, and bowel graft rejection. Subjects include warm-blooded animals, such as mammals, such as a primate, and, more preferably, a human. Non-human primates are subjects as well. The term subject includes domesticated animals (such as cats, dogs, etc.), livestock (for example, cattle, horses, pigs, sheep, goats, etc.) and laboratory animals (for example, mouse, rabbit, rat, gerbil, guinea pig, etc.). A subject, in some embodiments, is one who or that experiences abdominal pain but does not have a diagnosis of a pathological condition in the intestinal tract; alternatively, the subject has a provisional diagnosis of a pathological condition. In some embodiments, the subject is being treated for the pathological condition at the time the biological sample is taken from the subject. In some embodiments, the subject is not yet being treated for the suspected pathological condition at the time a sample is taken from the subject.

As used herein, “biological sample” refers to a composition containing a material for detection using the instant methodology, and includes, e.g., “biological samples”, which refer to any material obtained from a living subject noted herein. In some embodiments, the subject is being screened for a pathological condition of the intestinal tract, such as intestinal ischemia (including acute mesenteric ischemia), necrotizing enterocolitis, inflammatory bowel disease, and bowel graft rejection. The biological sample can be in any form, including a solid material such as a tissue, cells, a cell pellet, a cell extract, a surgical sample, a biopsy or fine needle aspirate, or it can be in the form of a biological fluid such as urine, whole blood, plasma, or serum, or any other fluid sample produced by the subject such as saliva or mucosa. In some embodiments, the biological sample is blood, plasma, or serum. In some embodiments, the biological sample is processed, e.g., to remove some components, e.g., by techniques to enrich components such as proteins by salt precipitation.

As used herein, “superparamagnetic (SPM) beads” refers to particles that are 10 nanometers to 30 micrometers in average diameter and including at least one material that makes them responsive to external magnetic fields, e.g., iron oxide nanoparticles. That is, and as described herein, in some embodiments, the present methodology employs superparamagnetic (SPM) beads sufficient to rapidly extract Villin-1 from a biological sample, such as bodily fluids like blood, plasma, or serum. Such extraction of Villin-1 makes it possible to enable the rapid identification of, for example, intestinal ischemia such as acute mesenteric ischemia (AMI).

Described herein are antibody surface coatings of the superparamagnetic beads as well representative ways for identifying binding of the analyte to the beads.

Antibodies having affinity for Villin-1 were identified using ELISA and surface plasmon resonance affinity biophysical measurements. In some embodiments, suitable antibodies have been immobilized on SPM beads and their surface coverage is optimizable for a desired level of sensitive analyte detection. In addition, in some embodiments, the surface coating is modifiable to produce a detectable signal that will allow the facile detection of SPM beads with high levels of sensitivity. This, in some embodiments, will enable Villin-1 to be rapidly detected at very high sensitivity levels directly from, for example, blood using flow cytometry, colorimetric assays, or lab on a chip technology.

“Threshold” as used herein refers to preselected reference levels of Villin-1. In some embodiment, the levels of Villin-1 measured in a sample are compared to a predefined reference threshold based on basal levels of Villin-1 in healthy population, in order to determine if the subject has AMI (level of Villin-1 in sample higher than the predefined threshold) or not (level of Villin-1 in sample lower than the predefined threshold). In another embodiment, the levels of Villin-1 measured in a sample are compared to predefined thresholds based on levels of Villin-1 associated with preselected degrees of severities, in order to evaluate the gravity of AMI-induced intestinal damages (with high levels of Villin-1 associated with advanced intestinal damages).

“Antibody” as used herein refers to an immunoglobulin, whether in its complete form or in fragments containing the immunologically active antigen-binding sites, such as the F(ab), the F(ab′)₂ or the F(ab′) fragments generated by enzymatic cleavage. The term antibody herein includes (but is not limited to) monoclonal, polyclonal, recombinant, humanized or chimeric antibodies as well as aptamers. The antibody can be unconjugated or conjugated with a range of biological/chemical compounds, including fluorophores (examples: rhodamine, AlexaFluor™ or fluorescein isothiocyanate [FITC]), haptens (example: biotin), enzymes (examples: horseradish peroxidase [HRP] or alkaline phosphatase [AP]), or nanoparticles. The antibody can be purchased from commercially available catalogues or produced by a range of methods (in-house or outsourced to companies specialized in antibody production). Herein, when it is stated that an “anti-XXX” antibody specifically binds to its target, it means that, when the antibody is added to a complex sample composed of a variety of proteins, cells, molecules (etc.), the antibody precisely binds to the epitope of its target “XXX”, corresponding to its antigen-binding site.

“Capture antibody” as used herein refers to any type of anti-Villin-1 antibody that is used to capture the Villin-1 in samples. It can be unconjugated or conjugated with various molecules (such as biotin). Depending on the methods, the capture antibody can be used in solution or be attached to a solid substrate (such as for example wells of a microplate or superparamagnetic beads).

“Detection antibody” as used herein refers to any type of anti-Villin-1 antibody that is used to measure the levels of Villin-1 present in samples. In some methods, it can directly be used for the evaluation of the levels of Villin-1 in samples (when the detection antibody is conjugated with nanoparticles or with fluorophores for example). In other methods, the addition of a substrate (such as for example 3,3′,5,5′-tetramethylbenzidine [TMB] when the detection antibody is conjugated with the enzyme HRP) could be used. In indirect measurement methods, a conjugated streptavidin (with HRP or fluorophore, for example) can be added when the detection antibody is conjugated with biotin), or a secondary antibody (conjugated with HRP or fluorophore for example) can be used when the detection antibody is conjugated to evaluate the levels of Villin-1 in samples.

“Secondary antibody” as used herein refers to any type of antibody specifically binding to the detection antibody (through for example specificity against the host specie of the detection antibody). It is used as an indirect way to measure the levels of Villin-1 present in samples in some of methods. It is normally conjugated with a biological/chemical compounds, including fluorophores or enzymes, that can be used to measure the levels of Villin-1 in samples.

“Control antibody” as used herein refers to any type of antibody specifically binding to the detection antibody (through for example specificity against the host specie of the detection antibody) in a lateral flow assay method for the evaluation of the levels of Villin-1. In this method, the control antibody is immobilised on the detection pad and capture the nanoparticles conjugated to the detection antibody (independently to the presence of Villin-1 samples) to form a control line on the lateral flow strip, that is used to confirm the validity of the lateral flow assay.

“Nanoparticles” as used herein refers to particles of small size (ranging from 10 nm to 30 μm). They can be constituted or coated with a range of compounds, including (but not limited to) gold, latex, iron oxide (in the case of superparamagnetic beads). In some methods, they can be further chemically modified to allow functionalization with protein (such as streptavidin) or with antibodies (such as anti-Villin-1 detection antibody), for example.

“Reader” as used herein refers to the different machines that can be used to evaluate the results of the methods used to determine the level of Villin-1 in samples. Readers include (but are not limited to) machine that can measure optical signals including colorimetric, fluorescent or chemiluminescent signals. The output can be qualitative, semi-quantitative or quantitative depending on the method used.

### Detection Methodology

As disclosed herein, the present disclosure contemplates a method for detecting and treating a pathological condition in a subject's intestinal tract, such condition chosen from intestinal ischemia (including acute mesenteric ischemia), necrotizing enterocolitis, inflammatory bowel disease, and bowel graft rejection. In some embodiments, the pathological condition of the intestinal track is intestinal ischemia, such as acute mesenteric ischemia.

Acute mesenteric ischemia or “AMI” as used herein refers to the obstruction of a blood vessel leading to hypoperfusion of the intestinal tissues, with consequences that comprise damages, necrosis and/or perforation of the intestinal tissues. More specifically, this invention applies to all the major types of AMI, including mesenteric arterial embolism, mesenteric arterial thrombosis, mesenteric venous thrombosis and nonocclusive mesenteric ischemia.

In some embodiments, the subject is a mammal, such as a primate, and, more preferably, a human. In some embodiments, the subject is a non-human primate. In some embodiments, the subject is chosen from domesticated animals, such as cats, dogs, etc., livestock (for example, cattle, horses, pigs, sheep, goats, etc.) and laboratory animals (for example, mouse, rabbit, rat, gerbil, guinea pig, etc.). The subject, in some embodiments, is one who or that experiences abdominal pain but does not yet have a diagnosis of a pathological condition in the intestinal tract; alternatively, the subject has a provisional diagnosis of a pathological condition. In some embodiments, the subject is being treated for the pathological condition at the time a biological sample is taken from the subject. In some embodiments, the subject is not yet being treated for the suspected pathological condition at the time a sample is taken from the subject.

In some embodiments, the subject is human, who may or may not have the pathological condition.

The method includes contacting a biological sample from the subject with a Villin-1 receptor and detecting binding between the Villin-1 receptor and Villin-1 when Villin-1 is present in the biological sample.

The biological sample, in some embodiments, is in a form of a solid, such as a tissue, cells, a cell pellet, a cell extract, a surgical sample, a biopsy or fine needle aspirate, or in some embodiments, the biological sample is in the form of a biological fluid such as urine, whole blood, plasma, or serum, or any other fluid sample produced by the subject such as saliva or mucosa. In some embodiments, the biological sample is in the form of feces. In some embodiments, the biological sample is blood, plasma, or serum. In some embodiments, the biological sample is processed, e.g., to remove some components, e.g., by techniques to enrich components, such as proteins, e.g., by salt precipitation.

“Villin-1” as used herein refers to the human Villin-1 protein, encoded by the VIL1 gene (located on the chromosome 2), a calcium-regulated actin binding protein, constituted of a large core (composed of repeated domains in its N-terminal) and of a small headpiece (in its C-terminal). The term Villin-1 incorporates the full protein, or only fragments of it. Similarly, it also includes any potential variants that could be produced by genetic diversities, by posttranslational modifications.

When Villin-1 is present in the biological sample in detectable amounts, the method detects binding between the Villin-1 receptor and Villin-1. In some embodiments, the detecting is calibrated to provide a method of determining whether Villin-1 is above or below a specified amount. Such information can be correlated with whether additional therapy (e.g., new or different drugs) is needed to improve the prognosis of the subject, thereby mitigating the risk associated with the pathological condition.

In some embodiments, detecting utilizes an assay device configured to use the Villin-1 receptor to assay Villin-1. In some embodiments, the assay device is chosen from dipstick, lateral flow, or flow-through devices, and in some embodiments, the assay device is configured to be an immunoassay.

In some embodiments, other methodology, such as competitive or inhibition assay, may be used for detecting Villin-1 in a sample.

In some embodiments, other methodology, such as competitive or inhibition assay, may be used for detecting α-glutathione S-transferase in a sample.

In some embodiments, other methodology, such as competitive or inhibition assay, may be used for detecting intestinal fatty acid binding protein (I-FABP) in a sample.

Although Villin-1 is the subject of the embodiments both above and below, it should be understood that the description applies to α-glutathione S-transferase and I-FABP.

In some embodiments, the Villin-1 receptor is chosen from anti-Villin-1 antibodies.

In some embodiments, the anti-Villin-1 antibody is C-terminus or N-terminus type.

In some embodiments, the anti-Villin-1 antibody is polyclonal, monoclonal or an antigen binding fragment thereof. In some embodiments, the anti-Villin-1 antibody is a modified antibody, such as a chimeric antibody, humanized antibody, or a fragment thereof. In some embodiments, the anti-Villin-1 antibody is synthetic, a single chain antibody, a single domain antibody, fragment variable (Fv), single chain Fv (scFv), etc.

In some embodiments, the anti-Villin-1 antibody is chosen from R814, e.g., Cell Signaling Technologies, SP145 from, e.g., Invitrogen, UMAB230 from, e.g., OriGene, OTI3B3 from OriGene 3E5G11 from, e.g., Abcam, EPR3490 from, e.g., Abcam, VIL1 from, e.g., Abbexa, AS1A11 from, e.g., G Biosciences, VIL1/1314 from, e.g., enquire BioReagents, 1D2C3 from, e.g., Santa Cruz Biotechnology, OAGA00811 from, e.g., Aviva Systems Biology, and OAEB02383 from, e.g., Aviva Systems Biology. In some embodiments, the anti-Villin-1 antibody is chosen from small chain antibody fragments, nanobodies and other genetically engineered protein receptors based on these antibodies may be suitable for this assay. Of course, and as known in the art, the application contemplates any antibody, aptamer, receptor, or the like that binds Villin-1. Suitable receptors may be identified by well-known techniques, such as phage display, and aptamers can be identified by well-known iterative selection procedures, such as SILEX.

In some embodiments, the anti-α-glutathione S-transferase antibody is chosen from: e.g., anti-α-glutathione S-transferase antibody, Merck, e.g., GSTA1/α-glutathione S-transferase antibody, BioOrbyt, e.g., anti-α-glutathione S-transferase antibody, Sigma, e.g., anti-α-glutathione S-transferase antibody, Abbexa, e.g., Glutathione S Transferase alpha 1 (GSTA1) Rabbit Polyclonal Antibody, Origene, e.g., GSTA1 Polyclonal Antibody, ThermoFisher Scientific, e.g., Glutathione S-Transferase alpha 3, Antibodies-online.com, e.g., Glutathione S-Transferase alpha, Cloud-Clone Corp, e.g., Glutathione S-transferase alpha, Boster Bio, e.g., Glutathione S-transferase alpha, Bio-Techne, e.g., Glutathione S-transferase alpha, Absolute Antibody, or, e.g., Glutathione S-transferase alpha, Cambridge Bioscience.

In some embodiments, the anti-I-FABP antibody is chosen from: e.g., Mouse anti-Human I-FABP/FABP2 Monoclonal Antibody (MBS246348), MyBioSource.com, e.g., Monoclonal Mouse anti-Human I-FABP/FABP2 Antibody, Lifespan Biosciences, e.g., anti-I-FABP antibody: Rabbit anti-Human I-FABP Polyclonal Antibody, MyBioSource.com, e.g., Mouse anti-Human FABP2 Monoclonal Antibody, ProteinTech, e.g., Fatty Acid Binding Protein 2, Intestinal (FABP2) Polyclonal Antibody, Biomatik, e.g., Recombinant Anti-I-FABP antibody, abCam, e.g., Rabbit Anti-Human FABP2/I-FABP pAb, Cell Sciences, e.g., Rat FABP2/I-FABP Biotinylated Antibody, R&D Systems, e.g., Rabbit Anti-Human FABP, Biorbyt, e.g., FABP2/I-FABP Antibody, Novus Biologicals, e.g., Intestinal Fatty Acid Binding Protein/I-FABP (FABP2) Antibody, Abbexa Ltd, e.g., Anti-FABP2 antibody, St. John's Laboratory, e.g., Anti-FABP2/I-FABP Antibody, BosterBio, e.g., Anti-FABP2, GeneTex, e.g., Anti-FABP2 Antibody, Rabbit Polyclonal, SionBiological, e.g., FABP2 antibody (Fatty Acid Binding Protein 2, Intestinal), Antibodies online, e.g., Rabbit Anti-FABP2, US Biological, e.g., FABP2 Polyclonal Antibody, Elabscience, e.g., I-FABP Polyclonal Antibody, G-Biosciences, e.g., I-FABP Antibody, Santa Cruz Biotechnology, Inc., e.g., I-FABP Antibody, Hycult Biotech, e.g., FABP2 Antibody, Thermo Fisher Scientific, e.g., FABP2 Antibody, NSJ Bioreagents, e.g., FABP2 Antibody, RayBiotech, e.g., FABP2 Antibody, AssayPro, e.g., FABP2 Antibody, Affinity Biosciences, e.g., FABP2 Antibody, OriGene Technologies, e.g., FABP2 Antibody, Cayman Chemical, e.g., FABP2 Antibody, Proteintech Group Inc, or e.g., FABP2 Antibody, ProSci.

In some embodiments, Villin-1 receptors are chosen from ligands that bind to Villin-1. In some embodiments, the ligands do not have a sequence of amino acids (non-peptide or non-protein).

In some embodiments, the Villin-1 receptors are unlabeled. In unlabeled embodiments in which the Villin-1 receptors are chosen from anti-Villin-1 antibodies, for example, the assay is an agglutination assay, which makes it possible to visualize the presence of the Villin-1 receptor by agglutination.

In some embodiments, the Villin-1 receptors are labeled. A detectable label usable in a method or apparatus described herein is not limiting as long as the label has a detectable physical or chemical property. Detectable labels have been developed in immunoassays. In some embodiments, the label can be, e.g., any composition detectable by spectroscopic, photochemical, biochemical, immunochemical, electrical, optical or chemical means. In some embodiments, the label is chosen from magnetic beads, fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horseradish peroxidase, alkaline phosphatase and others that can be used in an ELISA), and colorimetric or particulate labels such as colloidal gold or colored glass or plastic (e.g., polystyrene, polypropylene, latex, etc.) beads.

The label can be coupled directly or indirectly to the Villin-1 receptor.

In some embodiments, the label is a radioactive label, which is detectable by a scintillation counter or photographic film as in autoradiography. In some embodiments, the label is a fluorescent label, which is detectable by exciting the fluorochrome with the appropriate wavelength of radiation and detecting the resulting luminescence, which is detectable by the eye visually, or with assistance of photon counters, photographic film, photodiodes, or electronic detectors such as charge-coupled devices (CCDs) or photomultipliers and the like. Similarly, in some embodiments, the label is an enzymatic label, detectable by providing the appropriate substrates for the enzyme and detecting the resulting reaction product. In some embodiment, the label is a colorimetric label, detectable by observing a color associated with the label. In some embodiments, such as a dipstick assay, conjugated gold may appear pink, while various conjugated beads appear the color of the bead (sometimes white).

A detectable signal, in some embodiments, is compared to a reference and/or correlated with a predetermined level corresponding to treatment recommendations. In some embodiments, the predetermined level is an early signal from the same subject whose biological sample was assayed. Calibration can use, e.g., recombinant Villin-1, such as Villin (VIL1) (NM_007127) Human Recombinant Protein from Origene. For example, in a non-competitive assay to detect the level of a villin-1 in a biological sample from a subject, a level of signal below a predetermined level may be correlated with a recommendation that further treatment is not recommended based on this information alone. A different signal correlated with a higher level may indicate that additional treatment (or changes to treatment) is advisable. Other ways to calibrate are possible.

Detection of signal in an assay, in some embodiments, is visual, but may also be performed using a reader to detect a signal. Such readers include, for example, automated plate readers, EIA readers, and the like. Readers can be used for semi-quantitative or quantitative determination of the concentration for tested analytes.

In some embodiments, detecting binding between the Villin-1 receptor and Villin-1 lasts a period of time less than 120 minutes, 60 minutes, 30 minutes, or 10 minutes, starting after initiating the step(s) for contacting the biological sample from the subject.

In some embodiments, detecting binding between Villin-1 and Villin-1 receptor is performed by an immunological assay, such as ELISA. In some embodiments, the immunological assay is performed using a capture agent in a given configuration.

In some embodiments, the method utilizes a capture agent immobilized on a substrate to assay an analyte. In some embodiments, the substrate is chosen from glass, polystyrene, polypropylene, polyethylene, dextran, nylon, amylases, natural and modified celluloses, polyacrylamides, agarose, and magnetite. The substrate has a structural configuration chosen from spherical, like beads; cylindrical, like a test tubes and rods; and planar, like sheets, dishes, test strips, in which the structural configuration allows the capture agent to bind to an analyte.

The method, in some embodiments, detects signal related to a capture agent/analyte complex. In some embodiments, the detecting is noncompetitive. In some embodiments, the detecting is competitive. Competitive is useable in the sense that the measurement is of something competing for binding with the target like Villin-1.

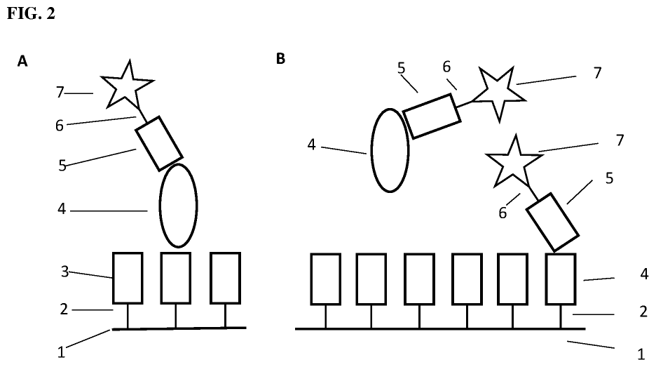

For example, FIG. 2A shows an example of a non-competitive immunoassay. In this illustrated non-competitive assay, a solid phase immunoassay, a capture agent 3 (e.g., an antibody Ab2) is immobilized on a solid phase substrate 1 (e.g., a plastic tube or beads), using physical adsorption (2 is absent) or covalent immobilization chemistry (2 is a bond or a linker). The biological sample, which could be either serum or plasma, contains Villin-1 4, which is reacted with the Villin-1 receptor 5 (e.g., a first antibody functionalized surface (Ab1)) that is labeled 7 (e.g., with a dye or biotin) after dilution in a buffer, such as phosphate buffer saline with Tween-20 (PBST). In the third phase of detection, capture agent 3 (Ab2) is used to immobilize the Villin-1/labeled Villin-1 receptor analyte (7,6,5,4) at the surface, where the analyte (7,6,5,4) is detected, e.g., using an amplification scheme, such as, fluorescence or enzyme enhanced colorimetric or chemiluminescence reactions. The excess sample or enzyme is removable by rinsing substrate 1 one or more times. In a non-competitive immunoassay the signal intensity is directly related to the concentration of the Villin-1.

For another example, FIG. 2B shows an example of a competitive immunoassay. In this illustrated competitive assay, a solid phase immunoassay, a capture agent 3 (e.g., an antibody (Ab1)) is immobilized on a solid substrate 1 and it used to indirectly detect the Villin-1 from the biological sample and in the presence of an analyte-a labeled Villin-1 receptor (e.g., an antibody (Ab2) 5 and enzyme 7 (E) that conjugates (Ab2-E).

The biological sample, which could be either serum or plasma, contains Villin-1 4, which is reacted with the Villin-1 receptor 5 (e.g., a first antibody functionalized surface (Ab1)) that is labeled 7 (e.g., with an enzyme E) after dilution in a buffer, such as phosphate buffer saline with Tween-20 (PBST). In the next phase of detection, capture agent 3 (Ab2) is used to immobilize the labeled Villin-1 receptor analyte (7,6) at the surface, where the analyte (7,6) is detected, e.g., using an amplification scheme, such as, fluorescence or enzyme enhanced colorimetric or chemiluminescence reactions. The excess sample or enzyme is removable by rinsing substrate 1 one or more times. In a non-competitive immunoassay the signal intensity is inversely related to the concentration of the Villin-1.

The following table helps clarify using numbers in parentheticals that refer to figure elements.

| | | | Measurement of |
|---|---|---|---|
| | Analyte | Capture agent | Villin-1 |
| Non- | Villin-1/labeled | second receptor for | Directly inferred |
| Competitive | Villin-1 receptor | Villin-1 (3) | |
| | (7, 6, 5, 4) | | |
| Competitive | Labeled Villin-1 | second receptor for | Inversely inferred |
| | receptor (7, 6, 5) | Villin-1 receptor (3) | |

(3 and 4) It is also possible to immobilize Villin-1 to the solid phase, which would allow the Villin-1 to be detected through either a non-competitive or competitive assay configuration.

As noted above, in some embodiments, a buffer is used for the immunological assays. In some embodiments, the buffer includes water and a surfactant, such as polysorbate surfactant such as TWEEN 20®, TWEEN 40®, TWEEN 60®, TWEEN 80®, SPAN 20®, SPAN 40®, SPAN 60®, SPAN 65®, and SPAN 80®. In some embodiments, the buffer is a buffered saline, such as Tris buffered saline (TBS) or phosphate buffered saline (PBS). In some embodiments, the buffer includes a blocking agent, for example a protein such as bovine serum albumin (BSA), milk or gelatin.

To be more valuable for urgent care applications, it is sometimes desirable that detection of the concentration of Villin-1 be completed in as short a time as possible. To increase the speed of analysis, it is sometimes desirable to use mass transfer reaction conditions that maximize the overall rate of reaction. Suspensions of microscopic particles makes it possible to increase rates of mass transfer and SPM beads allow these particles to be rapidly collected.

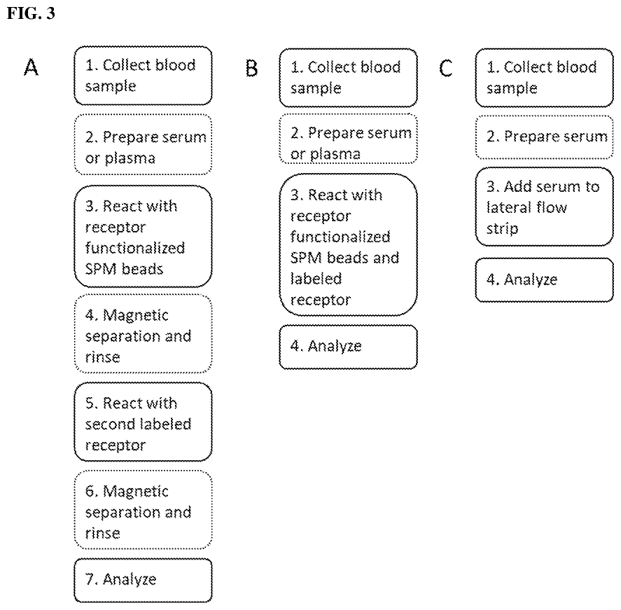

In some embodiments, detecting binding between Villin-1 and the Villin-1 receptor is performed using a magnetic bead assay (MBA), as outlined in FIG. 3A. A blood sample containing the analyte (Villin-1) is collected (FIG. 3A1). In some embodiments, the collected biological sample is first prepared for analysis by removing the blood cells resulting in serum or plasma. (FIG. 3A2). Here, the cell free biological sample is diluted in a solution that reduces nonspecific protein adsorption on the beads, e.g., phosphate buffer with a nonionic surface such as Tween20. In some embodiments, the diluted sample is incubated with anti-Villin-1 antibody (Ab1 (or other Villin-1 receptor)) functionalized SPM beads (FIG. 3A3). A magnetic field gradient is applied to the bead suspension, to separate the beads from biological sample and resuspend in a solution that may contain a second antibody (Ab2) (FIG. 3A5-6). Ab2 may be conjugated to chemical groups or nanoparticles that may be detected using chemical or physical means. The beads may then be analyzed for changes in physical or chemical properties. (FIG. 3A7) In some embodiments, a flow cytometer (FC) is used to monitor changes optical properties, i.e., size or fluorescence, making it possible to rapidly analyze 100,000 particles in 5 minutes. Alternatively, a micro reader may be used to measure the chemical properties of the solution from which the beads have been removed.

In some embodiments, several steps in the process may be eliminated (compared to FIG. 3A) by using specific receptors or analysis techniques. For example, FIG. 3B shows an assay in which the washing steps have been eliminated by using receptors that do not cross-react with serum and an analysis system that does not require separation.

In some embodiments, a lateral flow assay is desirable and has an analogous set of steps of collecting, preparing, and analyzing in FIG. 3C (1-2 & 4) but the biological sample is added to a later flow assay (FIG. 3C3).

In some embodiments, a lateral flow apparatus includes a sample receiving zone, a label zone, a test zone, and a control zone.

In some embodiments, the sample receiving zone accepts a fluid biological sample that Villin-1. A label zone is located downstream of the sample receiving zone, and contains one or more labeled reagents that recognize, or are capable of binding, to Villin-1 receptor. Further, a test region is disposed downstream from the label zone and contains test and control zones. The test zone(s) generally contain a capture agent associated with the substrate at the test zone. In some embodiments, the capture agent is immobilized on the substrate at the test zone. In general, the immobilized capture agent specifically binds to the analyte of interest.

As the fluid sample flows along the substrate, the analyte of interest binds with a mobilizable labeled reagent in the label zone, and then becomes restrained in the test zone. In some examples, the test region, when a control zone comprising a mark on the device is utilized, this mark is positioned about the test region such that it becomes visible within the test region when the test region is moist.

In some embodiments, the fluid biological sample flows along a flow path running from the sample receiving zone (upstream), through the label zone, and then to the test and control zones (downstream).

In some embodiments, the test device is configured to perform an immunological assay. In some instances, the liquid transport along the substrate is based upon capillary action. In another situation, the liquid transport along the substrate is based on non-bibulous lateral flow, wherein all of the dissolved or dispersed components of the liquid sample are carried at substantially equal rates and with relatively unimpaired flow laterally through the substrate, as opposed to preferential retention of one or more components as would occur, e.g., in materials that interact, chemically, physically, ionically or otherwise with one or more components.

In some embodiments, the labeling zone of immunoassay assay can also include control-type reagents. These labeled control reagents often comprise detectable moieties that will not become restrained in the test zones and that are carried through to the test region and control zones by fluid sample flow through the device. In some instances, these detectable moieties are coupled to a member of a specific binding pair to form a control conjugate that can then be restrained in a separate control zone of the test region by a corresponding member of the specific binding pair to verify that the flow of liquid is as expected. The visible moieties used in the labeled control reagents can be the same or different color, or of the same or different type, as those used in the analyte of interest specific labeled reagents.

In some embodiments, the method further includes obtaining a biological sample from the subject.

In some embodiments, the subject is being screened for a pathological condition of the intestinal tract, such as intestinal ischemia, necrotizing enterocolitis, inflammatory bowel disease, and bowel graft rejection.

### Superparamagnetic Beads

In some embodiments, polymer-coated superparamagnetic beads are coated with specific chemistries such that the particles have the ability to bind the corresponding targets from a mixture of biological materials. In some embodiments, these superparamagnetic beads are separated from the mixture by being attracted to an external magnetic field such that the target bound to the particle surface is separated. Paramagnetism occurs in the presence of an externally applied magnetic field, so superparamagnetic materials do not retain a significant amount of magnetization in the absence of an externally applied magnetic field. The ability to bind specific biological materials to the magnetic particles provides a simple and effective means for concentration, separation, and/or purification of targets.

Superparamagnetic beads are makeable by methods of U.S. Pat. No. 8,715,739, issued May 6, 2014, to G. Lee, which is incorporated herein by reference in its entirety. Briefly, a variety of superparamagnetic can be assembled and functionalized. While a variety of methods are known for forming such superparamagnetic particles, in some embodiments, magnetic particles can be prepared from core domains made by the co-precipitation of ferric and ferrous salts according to the method described by Landfester, “Magnetic Polystyrene Nanoparticles with a High Magnetite Content Obtained by Miniemulsion Processes”, Macromolecular Chemistry and Physics, 204, 2003, pp 22-31 and by U.S. Pat. No. 5,648,124. A mixture of ferrous chloride and ferric chloride in deoxygenated water is combined with aqueous ammonium hydroxide and heated with vigorous stirring. The resulting black slurry is then dialyzed, filtered, and hydrophobized with oleic acid.

In some embodiments, the superparamagnetic beads are made with one or more magnetic materials, such as particles including or consist essentially of a magnetic material such as Fe (including magnetite and maghemite), Ni, and Co, or mixtures of these materials. In other embodiments magnetic alloys, such as alloys containing Mn, and/or antimony, may be used. In some embodiments, ferrite particles (Fe₃O₄) or magnetite or maghemite are made.

In other embodiments, at least some of the superparamagnetic particles are made with one or more metallic materials, such as silver or gold.

In other embodiments, the superparamagnetic particles are made with one or more nonmetallic materials, such as a powder of oxides, e.g., silica.

In some typical embodiments, the superparamagnetic particles have core nanoparticles, optionally spherical and/or monodisperse. The average size of the nanoparticles is, in some embodiments, in the range of 1 nm to 100 nm or from 5 nm to 50 nm.

In some embodiments, a cluster of these core particles/nanoparticles are coated with a polymer like polystyrene or some other functionalizable coating. The resultant coated particles are typically 0.010 to 30 micrometers or from 0.100 to 3.0 micrometers or from 0.5 to 2.0 micrometers.

In some embodiments, the coating of the clusters is made with the methods of U.S. Pat. No. 8,715,739, issued May 6, 2014, to G. Lee, which is incorporated herein by reference in its entirety. In some embodiments, superparamagnetic particles/beads have a core that is substantially bare superparamagnetic nanoparticles free from polymer coating.

Such superparamagnetic particles make it possible to provide magnetically responsive microparticles having a core comprising nanoparticles which are superparamagnetic and exhibit negligible residual magnetism. Such nanoparticles may be made of, e.g., magnetite, and optionally may be less than 50 nm in size, and may exhibit only paramagnetic properties.

Villin-1 binds with antibodies functionalized on SPM beads and the magnetic field makes it possible to concentrate for detection, such as surface detection, or separate or purify.

The demonstration described below was performed with 1 micron diameter streptavidin beads produced according to U.S. Pat. No. 8,715,739 and coated with polyethylene glycol brushes. The assay has also been performed with streptavidin coated DYNABEAD M-270 streptavidin beads. The beads could be pre-functionalized with the antibodies or the antibodies could be mixed with the beads during the reaction.

### Bead Surface Chemistries

Several strategies can be used to functionalize the SPM beads with antibodies for this assay. It, in some embodiments, is desirable for this application to use a polymer layer between the bead surface and antibodies to minimize nonspecific adsorption of proteins present in the sample and maximize the reactivity of the antibodies. The antibodies, in some embodiments, are either covalently linked to the surface of the beads using bioconjugation chemistry or noncovalently linked to the beads using molecular recognition, such as, the well know streptavidin-biotin system. Below is described the polymer chemistries and bioconjugation chemistry.

The bare SPM beads are produced with a monolayer of chemical groups, e.g., carboxyl groups linked to a polymer, or with inert surface chemistry, i.e., silica. These beads in some embodiments, are coated with a monolayer of hydrophilic polymer by either grafting a polymer to the substrate or to react monomers with the surface of the bead to form the polymer film. The grafting of 2,000-20,000 molecular weight linear polyethylene glycol (PEG) to SPM beads has been described previously (H. Shang and G. U Lee, Magnetic Tweezers Measurement of the Bond Lifetime-Force Behavior of the IgG-Protein A Specific Molecular Interaction, Journal of the American Chemical Society 129, 6640-6646, 2007; G. U Lee, S. W. Metzger, M. Natesan, C. Yanavich, and Y. F. Dufrene, Implementation of Force Differentiation in the Immunoassay, Analytical Biochemistry 287, 261-271, 2000). In the ‘graft-to’ chemistry the surface of the beads reacted with a hetro-bifunctional linear PEG that bares a n-hydroxy succinimide (NHS) group and protected group at opposing ends. This allows the PEG to be grafted to the bead in aqueous solutions. A protein can then be grafted to the end of the PEG that has been protected. This chemistry has been shown to be efficient in minimizing nonspecific adsorption of proteins and allows antibodies, proteins, or biotin to be grafted to the surface using bioconjugation techniques (described herein). However, the hetrobifunctional polymer is relatively expensive and the graft-to polymer density is limited by the steric forces associated with a solvated polymer.

Polyethylene glycol brushes, in some embodiments, are reacted at the surfaces of catalyst functionalized silica coated beads using radical polymerization of the monomer, such as, polyethylene glycol methyl ether methacrylate. One such protocol is described below in detail:

Synthesis of the initiator: 3-(2-Bromoisobutyramido)propyl(triethoxy) silane (BiBB-APTES) was synthesized via a procedure previously described as follows. 30 ml (0.37 mol) of anhydrous THF was added to a solution of 4.68 mL (20 mmol) (3-aminopropyl)triethoxysilane and 3.35 ml (24 mmol) triethylamine in a 2-neck round bottom flask (RBF). RBF was sealed and placed in an ice bath. Reaction solution was stirred and purged with nitrogen for 10 minutes. 3 ml (24 mmol) of α-bromoisobutryl bromide was added dropwise over 10 minutes under ice cooling. Reaction solution allowed to return to room temperature and stirred under nitrogen flow for 3 h. Centrifugation (8000 RPM for 30 minutes), followed by removal of supernatant, was used to remove triethylammonium bromide after reaction. Rotary evaporation was used to remove any excess solvent. The solution was left under nitrogen atmosphere overnight which led to the formation of yellowish oil. (5.72 g, 15.46 mmol, 77%, ρ=1.04 g/cm³). ¹H NMR (400 MHZ, CDCl₃, δ): 3.7 (m, 6H), 3.3 (s, 2H), 1.9 (s, 6H), 1.65 (s, 2H), 1.2 (dt, 9H), 0.75 (s, 2H)

Reaction of BiBB-APTES with beads: 210 mL (2.59 mol) of tetrahydrofuran and 56.7 μL (0.99 mmol) of acetic acid were added to a flask containing the silica coated SPM beads. The reaction mixture was sealed in the flask and placed in the oil bath, preheated to 37° C. Solution was stirred slowly for 1 h at 37° C. 800 μL (2.25 mmol) of previously synthesized BiBB-APTES was mixed with 5 mL THF and added to the reaction solution. Reaction was heated to reflux at 75° C. for 2.5 h. Then, the functionalized beads were cleaned by washing with ethanol and DI water.

Polymerization: 48 mL MeOH and 96 mL DI water (both pre-purged with N₂) were added to a pre-purged solution of 72 g (0.14 mol) PEGMEMA, 2.8 g (17.9 mmol) 2,2′-bipyridyl (bpy) and initiator functionalized beads under N₂. The clear solution was swirled and sonicated until homogenous. Then, 514 mg CuBr (3.58 mmol) was added under N₂ before 5 minutes of sonication. The dark brown monomer solution was transferred to the purged reactor in an oil bath, preheated to 60° C. 34 μL (95.46 μmol) BiBB-APTES was added as a sacrificial initiator and the polymerization proceeded for a defined reaction time. Subsequently, the reaction mixture was removed, the substrates placed in a beaker of MeOH and sonicated for 5 minutes to remove any physisorbed, free-polymer. Substrates were rinsed thoroughly with methanol and finally stored in fresh MeOH at 4° C. Polymer coated beads were characterized using optical microscopy and dynamic light scattering.

The protein functionalized beads are created by coupling the antibody or streptavidin to the surface of the SPM bead using a covalent bioconjugation chemistry. In the case of the surfaces that are functionalized with the graft-to PEG polymers this is achieved by deprotecting the PEG monolayer and then reacting the protein with the beads in the presence of the appropriate crosslinker. For example, if the PEG is terminated with a carboxyl group it can be crosslinked to the primary amine on a protein, i.e., the anti-Villin-1 antibody or streptavidin, using 1-ethyl-3-(3-dimethylaminopropyl)-carbodiimide hydrochloride (EDAC).

In the case of the free radical polymerization from the surface of the bead the PEG brush polymer is end terminated with a bromine group. This group can be easily modified with an azide to allow ‘click’ chemistry to be used to immobilize the protein. That chemistry is described next.

Substitution of bromine end-group functionality 100 mg (1.25 mmol) of sodium azide was added to a Petri dish containing 20 mL (0.26 mol) dimethylformamide which contained the PEGMEMA-functionalized substrates. The dish was sealed with parafilm and placed on an orbital shaker at 300 RPM for 18 hours at room temperature. Substrates were cleaned with ethanol and blown dry before being stored under nitrogen for usage.

Functional the protein with dibenzocyclooctyne (DBCO): 4.45 mM protein was added to a solution of 26.4 μL (7 mM) NHS-PEG₄-DBCO and 48.6 μL PBS (1λ, pH 7.3) in a low-bind Eppendorf tube. Tube was placed on a rotating wheel for 17 h at room temperature. The excess NHS-PEG₄-DBCO was removed by size exclusion chromatography.

The demonstration below uses the streptavidin functional 1 μm diameter beads (U.S. Pat. No. 8,715,739) coated with polyethylene glycol brushes, a controlled density of streptavidin, and uses biotin to link antibodies to these surfaces. This results in a bead that has a controlled density of functional receptors and resists nonspecific protein adsorption. Streptavidin coated DYNABEAD M-270 beads have also been used but require multiple rinsing steps, as in FIG. 3A.

### Pathology Detection and Treatment

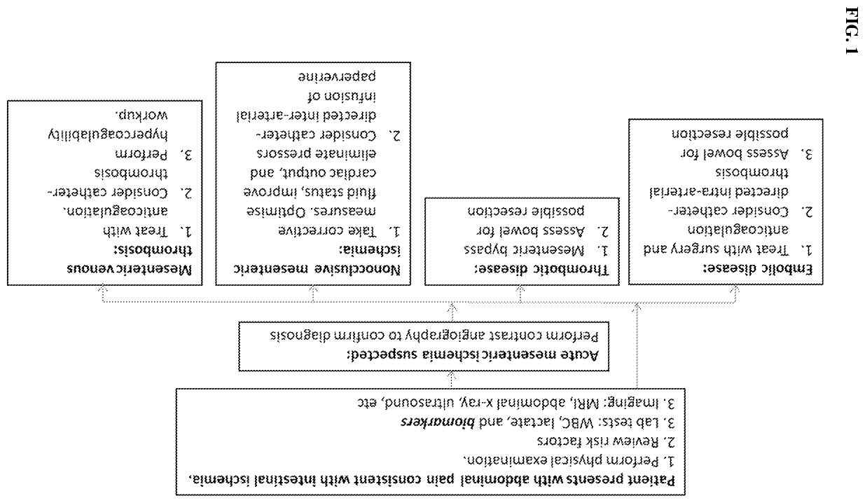

The present application contemplates methodology for detecting and treating a pathological condition, such as intestinal ischemia, in a subject's intestinal tract. FIG. 1 shows relevant information for a subject suspected of having a pathological condition.

### Intestinal Ischemia

In some embodiments, the pathological condition is intestinal ischemia. In some embodiments, the subject has one or more symptoms chosen from abdominal pain; an urgent need to have a bowel movement; frequent, forceful bowel movements; abdominal tenderness or distention; blood in the subject's stool; and mental confusion. In some embodiments, the subject has abdominal pain.

In some embodiments, the method thereafter comprises initiating or changing treatment of intestinal ischemia, such as acute mesenteric ischemia, comprising administering to the diagnosed subject an effective amount of one or more medicines for the treatment of intestinal ischemia such as acute mesenteric ischemia. In some embodiments, the one or more medicines comprise one or more anticoagulants, such as those chosen from warfarin, heparin, rivaroxaban (Xarelto) dabigatran (Pradaxa) apixaban (Eliquis), and edoxaban (Lixiana). In some embodiments, the one or more medicines comprises antibiotics, such as those chosen from Penicillins, such as penicillin V potassium, amoxicillin, amoxicillin/clavulanate (Augmentin); Tetracyclines, such as doxycycline, tetracycline, and minocycline; Cephalosporins, such as cefuroxime (Ceftin), ceftriaxone (Rocephin), and Cefdinir (Omnicef); Quinolones, such as ciprofloxacin (Cipro), levofloxacin (Levaquin), and moxifloxacin (Avelox); Lincomycins, such as clindamycin (Cleocin) and lincomycin (Lincocin); macrolides, such as azithromycin (Zithromax), clarithromycin (Biaxin), and erythromycin; Sulfonamides, such as sulfamethoxazole-trimethoprim (Bactrim, Bactrim DS, Septra), sulfasalazine (Azulfidine), and sulfisoxazole (optionally combined with erythromycin); Glycopeptides, such as dalbavancin (Dalvance), oritavancin (Orbactiv), telavancin (Vibativ), and vancomycin (Vancocin); Aminoglycosides, such as gentamicin, tobramycin, and amikacin; and Carbapenems, such as imipenem/cilastatin (Primaxin), meropenem (Merrem), doripenem (Doribax), and ertapenem (Inanz). In some embodiments, the antibiotics are chosen from aminoglycosides, ampicillin, amoxicillin, amoxicillin/clavulanic acid (Augmentin), carbapenems (e.g., imipenem), piperacillin/tazobactam, quinolones (e.g., ciprofloxacin), tetracyclines, chloramphenicol, ticarcillin, trimethoprim/sulfamethoxazole (Bactrim), doxycycline, cephalexin, clindamycin, metronidazole, azithromycin, and levofloxacin. In some embodiments, the antibiotics are chosen from broad spectrum antibiotics.

### Necrotizing Enterocolitis

In some embodiments, the condition is necrotizing enterocolitis. In some embodiments, the subject has one or more symptoms chosen from abdominal distention (bloating or swelling); feedings stay in the stomach instead of moving through to the intestines as normal; bile-colored (greenish) fluid in the stomach; bloody bowel movements; and signs of infection such as apnea (stopping breathing), low heart rate, lethargy (sluggishness).

In some embodiments, the method thereafter comprising initiating or changing treatment of necrotizing enterocolitis, comprising administering to the diagnosed subject an effective amount of one or more medicines for the treatment of necrotizing enterocolitis. In some embodiments, one or more medicines comprises antibiotics, such as those described herein.

### Inflammatory Bowel Disease

In some embodiments, the condition is inflammatory bowel disease, such as ulcerative colitis or Crohn's disease. In some embodiments, the subject has one or more symptoms chosen from diarrhea, abdominal pain, fatigue and weight loss.

In some embodiments, the method thereafter comprises initiating or changing treatment of inflammatory bowel disease, comprising administering to the diagnosed subject an effective amount of one or more medicines for the treatment of inflammatory bowel disease.

In some embodiments, the one or more medicines are chosen from antibiotics, anti-inflammatory drugs, and immune system suppressors. In some embodiments, the one or more medicines comprises antibiotics, such as those described herein.

In some embodiments, the one or more medicines comprises anti-inflammatory drugs, such as corticosteroids and aminosalicylates. In some embodiments, the anti-inflammatory drugs are chosen from mesalamine (such as Asacol HD, Delzicol, and others), balsalazide (Colazal) and olsalazine (Dipentum). In some embodiments, the one or more medicines comprises immune system suppressors, such as TNF-alpha inhibitors and biologics. In some embodiments, the immune system suppressors are chosen from infliximab (Remicade), adalimumab (Humira), golimumab (Simponi), natalizumab (Tysabri), vedolizumab (Entyvio), and ustekinumab (Stelara).

### Bowel Graft Rejection

In some embodiments, the condition is bowel graft rejection. In some embodiments, the subject has no symptoms, or one or more symptoms chosen from fever, malaise, change in ostomy output (increased or decreased), intestinal bleeding, nausea, and vomiting.

In some embodiments, the method thereafter comprises initiating or changing treatment of bowel graft rejection, comprising administering to the diagnosed subject an effective amount of one or more medicines for the treatment of bowel graft rejection different than earlier treatments.

In some embodiments, one or more medicines are chosen from antibiotics and anti-rejection medicines. In some embodiments, one or more medicines comprises antibiotics, such as those described herein. In some embodiments, the one or more medicines comprises anti-rejection medicines, such as immunosuppressive agents. In some embodiments, the anti-rejection medicines are chosen from Tacrolimus (Prograf), Sirolimus (Rapamune), Steroids, and Everaloums.

### Villin-1 Detection Kit

The present application contemplates a biomarker-detection kit for an emergency care or clinical laboratory that is composed of a sample preparation chamber for the collection of blood for analysis of Villin-1; reagents for the detection of Villin-1, e.g., the anti-Villin-1 functionalized superparamagnetic beads and detection anti-Villin-1; and a detection system for the detection of the reaction with Villin-1 from the biological sample. Two workflows for the operation of this kit are presented in FIGS. 3A and B in which magnetic separation and buffer exchange are and are not use, respectively.

The blood collection chamber may be selected to remove specific cellular and proteins from blood to improve the sensitivity or specificity of detection of Villin-1. This includes the preparation of serum or plasma from blood using silica tubes or tubes containing EDTA or heparin. This sample may also be prepared for analysis by centrifugation, filtration or affinity separation. The serum or plasma may also be diluted, and surfactants may be added to improve the binding efficiency of the receptors, adsorption of unwanted materials on the SPM beads, and/or reduce aggregation of the SPM beads.

The kit, in some embodiments, has a chamber containing one or more protein or nucleic acid receptors for the capture and detection of Villin-1. At least one of these receptors 3 will be used to specifically capture Villin-1 4 from the blood sample, as presented schematically in FIG. 2. In the sandwich assay format (FIG. 2A), Villin-1 is captured on a solid matrix 1, e.g., superparamagnetic bead or nitrocellulose membrane, functionalized with a chemical layer that minimizes nonspecific interactions 2. Villin-1 is detected with a second Villin-1 receptor 5 that binds to a separate epitope on Villin-1. The immobilized capture agent receptor 3 to the solid matrix should be achieved in a means that minimizes unwanted interactions with the blood sample, e.g., a PEG monolayer, may be used to reduce the binding of serum proteins to the solid matrix. A rinsing step may also be used between the capture and detection steps to increase the specificity of the detection Villin-1 receptor 5 with Villin-1.

In the indirect assay format (FIG. 2B), Villin-1 4 will be captured by the Villin-1 receptor 5. This will block the interaction of this Villin-1 receptor (E) 5 with the capture agent receptor 3 on the solid matrix substrate 1, e.g., superparamagnetic bead or nitrocellulose membrane, functionalized with a chemical layer that minimizes nonspecific interactions 2.

Detection of Villin-1 is performed by a number of direct or indirect chemical or physical means, as described in the examples below. Direct detection, in some embodiments, is performed by conjugating an optically active group label 7, e.g., fluorescein, green fluorescent protein, rhodamine, cyanine dyes, Alexa dyes, luciferase, quantum dots, magnetic beads, radiolabels, among others, to receptor 5 for detection using optical means, e.g., flow cytometer or fluorometer. Indirect detection can be performed by conjugating an enzyme label 7, e.g., horse radish peroxidase or alkaline phosphatase, to Villin-1 receptor 5 that rapidly reacts with specific chemical groups to produce an optical signal.

The Villin-1 assay may also be deployed in a format that will allow it to be used at the point of care. For example, lateral flow assays may be used to detect the analyte using the two receptors described in FIGS. 2 and 3. In this case, the capture agent 3 and substrate 1 is a nitrocellulose membrane and detector label 7 is colloidal gold, although many other nanometres scale detection technologies have been described in the literature.

The kit, in some embodiments, contains control formulations (positive and/or negative). This can be achieved by immobilizing reagents for these controls on a microscope particle, such as, a SPM beads, that have a specific size or color. Alternatively, a solid substrate, such as, porous nitrocellulose strip, can be functionalized with several biomarker detection site. The measurement or detection region of the microscopic beads or porous strip may include a plurality of antibody or nucleic acid receptors.

Instructions for carrying out the assay may also be included in the kit.

In one embodiment, the invention herein provides a novel screening test to diagnose AMI in a subject presenting with non-specific symptoms. To do that, a variety of biochemical, molecular and immunological methods can be used to evaluate the levels of Villin-1 in a sample from a subject. The comparison of the levels of Villin-1 measured with a predefined diagnostic threshold is indicative of the absence (lower than the threshold) or the presence (higher than the threshold) of AMI in the subject.

In another embodiment, the invention herein provides a staging test to assess the severity of AMI-induced intestinal damages in a subject diagnosed with AMI. To do that, a variety of biochemical, molecular and immunological methods can be used to precisely measure the level of Villin-1 in a sample from a subject. The comparison of the levels of Villin-1 with predefined severity thresholds (associated with known degrees of intestinal damages) indicates what is the degree of AMI-induced intestinal damages in the subject (with higher levels of Villin-1 associated with severe intestinal damages, including potential necrosis or perforation of the intestine).

In another embodiment, the invention herein provides a new strategy to track treatments efficacity and subject recovery. To do that, a variety of biochemical, molecular and immunological methods can be used to precisely measure the level of Villin-1 in a sample from a subject during and after completion of the treatments. The comparison of those levels of Villin-1 with the initial levels of Villin-1 in the same subject can ensure, respectively, that the treatments provided are efficient and that the subjects are fully recovered (with reduction of the levels Villin-1 up to being present at lower levels than the diagnostic threshold). Levels of Villin-1 remaining high is the sign of failure of the treatments and/or of additional intestinal damages, therefore, other treatment options (including surgeries) should be rapidly given to the subject.

Therefore, the invention herein includes the different biochemical, molecular and immunological methods able to qualitatively, semi-quantitatively and/or quantitatively evaluate the levels of Villin-1 in samples, for the use as novel screening, staging and prognosis tests for subjects affected by AMI. To only cite a few, methods that can be used include ELISA, magnetic bead assays, lateral flow assay or magnetic lateral flow assay. The methods in the invention also comprise any kits constituted of at least one anti-Villin-1 antibody and kits using a panel of biomarkers for AMI, including at least one anti-Villin-1 antibody and at least one antibody targeting one of the following: I-FABP, D-lactate, L-lactate, D-dimer, citrulline that are used to either diagnose AMI, stage AMI-induced intestinal damages or monitor recovery in subjects.

### General Methodology

These sections describe some of the biochemical, molecular and immunological methods that can be used to evaluate the levels of Villin-1 in samples of subjects. These methods are examples and are not limiting the scope of the methods that can be used in this invention. In general, the methods rely on the use of at least one anti-Villin-1 antibody. According to the methods, the outputs have different form, either qualitative, semi-quantitative or quantitative measurement of the levels of Villin-1 in samples that are compared to predefined diagnostic threshold for the diagnosis of AMI, to predefined severity thresholds for the assessment of potential AMI-induced pathologies and to levels of Villin-1 measured in prior samples collected from the same subject for the evaluation of the recovery of the subject.

### Subjects, Samples and Sample Preparation

In some embodiments of the invention, the subjects include persons arriving at a healthcare facility with unspecific symptoms including sudden-onset abdominal pain. In those, a sample is collected (and processed as required), and levels of Villin-1 are promptly evaluated to allow for rapid diagnosis of AMI by comparison with a predefined threshold. If the measured levels of Villin-1 are below the threshold, the diagnosis for AMI is negative and additional tests are required for diagnosis of the condition. At the opposite, if the measured levels of Villin-1 are superior to the threshold, the subject is diagnosed with AMI and best medical care will be given as deemed appropriate by the ones with skills in the art.

In some other embodiments, a sample will be collected (and processed as required) in subjects diagnosed with AMI for the precise evaluation of the levels of Villin-1 to determine the severity of the AMI-induced intestinal damages by comparison with predefined severity thresholds. This information will facilitate the choice of best treatment, in particular regarding the need for surgery due to the presence of necrotic tissues.

In some embodiments, a sample will be collected (and processed as required) in the same subject throughout the treatment and recovery phases. The levels of Villin-1 are determined in order to compare those levels with initial measurements. These repeated measurements can ensure that no additional intestinal tissue damage is present in the subjects and that healing is successful. The presence of levels of Villin-1 higher than anticipated, will be the signal that additional surgery might be required. This is especially true when the condition is acute mesenteric ischemia.

In all embodiments, standardized sample collection protocols and standardized processing protocols (if required) are followed to ensure consistence, replicability and reliability of the results obtained by measurement of the levels of Villin-1 for the diagnosis of AMI, its staging and assessment of recovery of all subjects.

### Lateral Flow Assay Methods for the Evaluation of the Levels of Villin-1

“Lateral flow assay” herein refers to a type of immunoassay in which capillary forces carries the sample through a series of pads with each specific characteristics and roles that allow to generate a measurable signal corresponding to the absence, presence or quantity of the analyte target in the sample. It has the crucial advantages of offering a rapid, affordable and easy to use method to analyze the levels of a specific target in a sample.

Whilst the principle of a lateral flow assay is well known for those in the art, we will provide some general information. A lateral flow strip is generally constituted of various pads, a sample pad, a conjugate pad, a detection pad and an absorbent pad assembled with specific overlap (to ensure continuity of the flow through the full strip) that are cut in strips of specific width, before being inserted in a housing cassette and stored in dry and cool conditions, usually in sealed bags alongside desiccants.

A wide range of buffers can be used in lateral flow assays, typically composed of buffering solutions with specific ionic strength (such as borate buffer, phosphate-buffered saline buffer, Tris buffer, and the like), blocking agents (such as bovine serum albumin BSA, casein, and the like), chaotropic agents (such as polyvinyl alcohol, and the like), compounds allowing to improve the immobilization and/or stability of antibodies (including proteins, sugars such as sucrose, lactose or trehalose, alcohols such as methanol, isopropanol or ethanol, and the like), detergents (such as Triton or Tween-20 . . . ) and preservatives (such as sodium azide, and the like). A key element of the good functioning of a lateral flow assay is the choice of the antibodies, which need to be specific to the target, to be stable at a range of temperature, humidity, pressure, and drying cycle (in term of structure and function), to have fast association kinetics (within seconds if possible, as there is almost no incubation time between the target in the sample and the antibodies), and to make strong bound with the target (to ensure no loss of signal due to the capillary flow). In most lateral flow assays, three antibodies are required (more complex lateral flow assays can include more antibodies, for example if more than one target is to be analyzed in a sample). Two of these antibodies are stripped on the detection pad, at position referred to as test line and control line for respectively, the capture antibody (specifically binding to Villin-1 in this method), and a control antibody (specifically binding to the detection antibody in this method). The third antibody, referred to as the detection antibody, is normally conjugated to nanoparticles and dried on the conjugate pad. A wide variety of nanoparticles can be used, with different characteristics, advantages and disadvantages. These include gold nanoparticles, latex nanoparticles, carbon-based nanoparticles/nanotubes, magnetic nanoparticles, among others. The conjugation of the detection antibody with the nanoparticles can be done through different methods, including passive conjugation (through adsorption of the antibody on the nanoparticle surface) or by covalent linkage (through for example EDC/sNHS-mediated binding between carboxylic acid on the nanoparticle surface with the primary amines on the antibody).

The invention herein includes all types of ELISA, irrespectively of their design (i.e., including, but not limited to, direct, indirect, competitive or sandwich ELISA with direct/indirect measurement, with capture antibody coated on plate or on superparamagnetic nanoparticles) and includes all types of sensing methods that can accurately measure the presence or the levels of Villin-1 in samples.

### Other Magnetic Beads Assays for the Evaluation of the Levels of Villin-1

A variety of other immunoassay working on very similar methods as ELISA can also be used to detect Villin-1 in samples. In these methods, the evaluation of the levels of Villin-1 is done using direct detection (by for example coupling the detection antibody) or by indirect detection (by for example coupling a secondary antibody) with compounds that can directly emit signal, including a fluorophore (such as FITC, rhodamine), a bioluminescent compound (such as luciferase) or radioactive compound. By comparison of the signal emitted in the sample with standard of known concentrations, it is possible to determine the levels of Villin-1 in the sample of subject to assist with diagnosis and prognosis of subjects.

The following Examples are illustrative and non-limiting.

### EXAMPLE 1: ANTIBODIES FOR THE DETECTION OF VILLIN-1

An enzymatic linked solid phase immunoassay (ELISA) was performed to evaluate the detection limit of Villin-1 for the anti-Villin antibodies described in this study

The following reagents were used:
i. Nunc Maxisorp 96-wellplate
ii. Villin-1 full length protein from OriGene
iii. Anti-Villin-1 3E5G11 (N-terminal monoclonal) from Abcam in PBS with 0.05% NaN₃
iv. Anti-Villin-1 R814 (polyclonal rabbit) from Cell Signaling Technologies in PBS with 1% BSA
v. Anti-mouse HRP dil 1/10000 (for the N-terminal Ab)
vi. Anti-Rb-HRP dil 1/60 (for the polyclonal Ab)
vii. BSA solution 2% by weight in PBS (1×, pH7.4)
viii. PBS (1×, pH7.4) and PBS-Tween20 (1×, pH7.4, 0.1% Tween)
ix. TMB (3,3′,5,5′-Tetramethylbenzidine) solution (colorimetric substrate for HRP)
x. H₂SO₄ solution 2 M (stop the colorimetric reaction)

### Procedure:

1. The 96-wellplate was incubated with different concentrations of Villin-1:0.005-500 ng/mL (4 wells per each concentration) overnight at 4° C. under shaking.
2. The plate was washed 3× with PBS and then incubated with 200 μL of 2% BSA solution in PBS (1×, pH7.4) for 3 h at 4° C.
3. From the 96-wellsplate the BSA solution was removed, and the wells were washed 3 times with PBST 0.1.
4. The solutions of the anti-Villin-1 antibodies at 6.4 μg/mL in PBS were added in duplicate to the wells (50 μL/well) containing the different concentrations of Villin-1 and to 4 extra wells blocked with BSA for control. The plate was shaken for 1 h at room temperature and then washed 3× with PBST 0.1%.
5. Solutions of the secondary antibodies were added to the wells containing the respective primary antibodies and to 4 extra wells blocked with BSA for control. The plate was covered with parafilm and aluminum foil and shaken for about 1h at room temperature and then washed with PBST 0.1%.
6. In all the wells 50 μL of TMB solution at room temperature were added and the plate was shaken for around 5 minutes covering the 96-well plate with the aluminum foil (the solutions turn to blue).
7. 50 μL of H₂SO₄ 2 M were added in each well to stop the reaction (the solutions turn to yellow).
8. The 96-well plate was read at 450 nm.

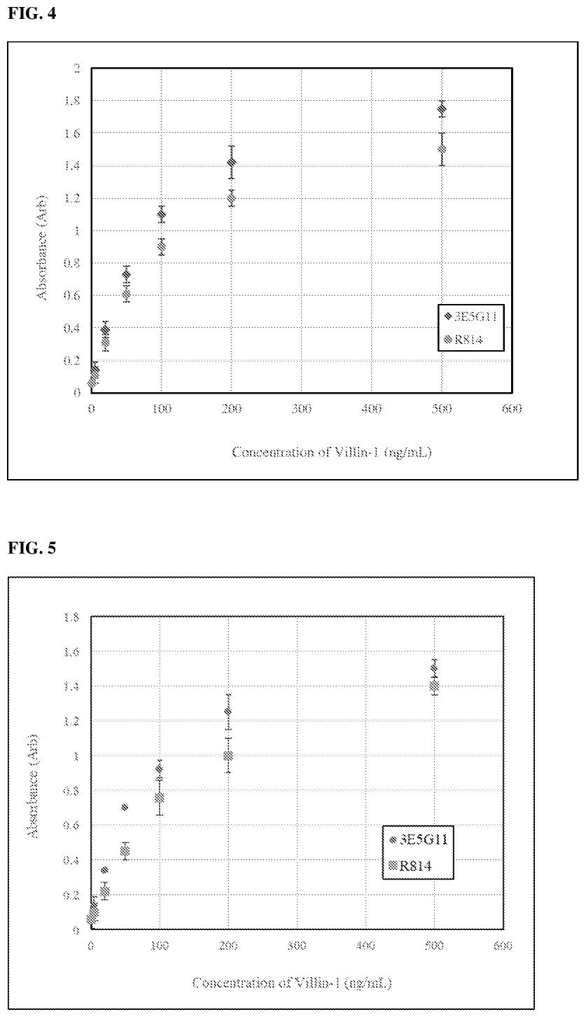

FIG. 4 presents absorbance data for the two different antibodies, i.e., 3E5G11 and R814, measured on the plates prepared with various concentrations of Villin-1. These absorbance measurements show that Villin-1 was detected until 1.0 ng/mL with a low background signal from the controls. Controls were run with both antibodies for wells prepared without Villin-1 and had adsorption values of 0.0405.

### EXAMPLE 2: SANDWICH ELISA FOR VILLIN-1 IN BUFFER

A non-competitive ELISA was performed on the recombinant Villin-1 (full length) with the two anti-Villin antibodies (N-terminal 3E5G11, polyclonal R814). This time either the N-terminal or the polyclonal antibody were adsorbed on the plate (Ab1). Then the plate was blocked with BSA before adding solutions at different concentration of Villin-1. Solution of the N-terminal antibody (Ab2) was added to the wells where the polyclonal antibody (Ab1) was previously adsorbed while a solution of polyclonal antibody (Ab2) was added to the wells with the N-terminal antibody (Ab1) adsorbed. A range of concentrations of the second antibody (Ab2) were also tested, i.e., 500, 1,000 and 2,500 ng/mL. After the washing steps, solutions of the secondary antibody HRP conjugate (i.e., anti-mouse-HRP and anti-Rb-HRP) were added to the wells containing the respective primary antibodies (Ab2).

The following reagents were used:
Nunc Maxisorp 96-wellplate
i. Villin full length protein from OriGene
ii. Anti-Villin 3E5G11 (N-terminal) from Abcam in PBS with 0.05% NaN₃
iii. Anti-Villin R814 (polyclonal rabbit) from Abcam in PBS with 1% BSA
iv. Anti-mouse HRP dil 1/10000 (for the N-terminal Ab)
V. Anti-Rb-HRP dil 1/60 (for the polyclonal Ab)
vi. BSA solution 2% in PBS (1×, pH7.4)
vii. PBS (1×, pH7.4) and PBS-Tween20 (1×, pH7.4, 0.1% Tween)
viii. TMB (3,3′,5,5′-Tetramethylbenzidine) solution (colorimetric substrate for HRP)
ix. H₂SO₄ solution 2 M (stop the colorimetric reaction)

### Procedure:

The wells were incubated with 5 μg/mL solution in PBS of 3E5G11 (50 μl/well) or 5 μg/mL solution in PBS of the R814 (50 μl/well) overnight at 4° C. under shaking.

The plate was washed 3× with PBS and then incubated with 200 μL of 2% BSA solution in PBS (1×, pH7.4) for 3 h at 4° C.

From the 96-well plate the BSA solution was removed and the wells were washed 3 times with PBST 0.1%.

Villin-1 solutions at different concentrations (0.005-500 ng/ml) in PBST 0.1% were added to the wells.

The plate was shaken for 1 h at room temperature and then washed 3× with PBST 0.1%.

The solutions of the two complementary anti-Villin-1 antibodies in PBST 0.1% were added in duplicate to the wells (50 μL/well) at 5 μg/mL.

The plate was shaken for about 40 min at room temperature and then washed 3× with PBST 0.1%.

Solutions of the secondary antibodies were added to the wells containing the respective primary antibodies and to 4 extra wells blocked with BSA for control (i.e., anti-mouse-HRP and anti-Rb-HRP). The plate was covered with parafilm and aluminum foil and shaken for about 30 min at room temperature and then washed with PBST 0.1%.

In all the wells 50 μL of TMB solution at room temperature were added and the plate was shaken for around 5 minutes covering the 96-well plate with the aluminum foil (the solutions turn to blue).

50 μL of H₂SO₄ 2 M were added in each well to stop the reaction (the solutions turn to yellow).

The 96-well plate was read at 450 nm.

FIG. 5 presents the absorbance values for the sandwich ELISA performed on different concentrations of Villin-1, i.e., 0.005-500 ng/mL, in PBST using R814 and 3E5G11 as the Ab1 antibody. FIG. 5 presents measurements showing that Villin-1 was detected at a sensitivity of 1.0 ng/mL for both R814 and 3E5G11 antibodies adsorbed on the plate (Ab1). The nonspecific background from control measurements without Ab1 and Ab2 was 0.0525 and 0.0465, respectively.

### EXAMPLE 3: NONCOMPETITIVE ELISA FOR VILLIN-1 IN HUMAN SERUM

A noncompetitive ELISA was performed to evaluate the detection efficiency of Villin-1 (full length) by the two anti-Villin antibodies (N-terminal: 3E5G11, polyclonal: R814) in serum. The assay was performed similar to Example 2 but this time only the polyclonal antibody was adsorbed on the plate (Ab1). Solutions of Villin-1 at different concentrations (0.005-500 ng/mL) and in different percentages of human serum (10, 25 and 50%) were tested.

The following reagents were used:
i. Nunc Maxisorp 96-wellplate
ii. Human serum from Sigma Aldrich
iii. Villin full length protein from OriGene
iv. Anti-Villin 3E5G11 (monoclonal N-terminal) from Abcam in PBS with 0.05% NaN3
v. Anti-Villin R814 (polyclonal rabbit) from Abcam in PBS with 1% BSA
vi. Anti-Rb-HRP dil 1/60 (for the R814 Ab)
vii. BSA solution 2% in PBS (1×, pH7.4)
viii. PBS (1×, pH7.4) and PBS-Tween20 (1×, pH7.4, 0.1% Tween)
ix. TMB (3,3′,5,5′-Tetramethylbenzidine) solution (colorimetric substrate for HRP)
x. H2SO4 solution 2 M (stop the colorimetric reaction)

### Procedure:

The wells were incubated with 5 μg/mL solution in PBS of polyclonal Ab (50 μl/well) overnight at 4° C. under shaking.

The plate was washed 3× with PBS and then incubated with 200 μL of 2% BSA solution in PBS (1×, pH7.4) for 3 h at 4° C.

From the 96-wellsplate the BSA solution was removed and the wells were washed 3 times with PBST 0.1%.

The Villin-1 solutions at different concentrations (0.005-500 ng/mL) and different percentage of serum (10, 25 and 50%) were added to the well. The plate was shaken for 1 h at room temperature and then washed 3× with PBST 0.1%.

The solutions of the N-terminal anti-Villin-1 antibody in PBST 0.1% were added in duplicate to the wells (50 μL/well).

The plate was shaken for about 40 min at room temperature and then washed 3× with PBST 0.1%.

Solution of the anti-Rb-HRP 1/60 in PBST 0.1% was added to all the used wells. The plate was covered with parafilm and aluminum foil and shaken for about 30 min at room temperature and then washed with PBST 0.1%.

In all the used wells 50 μL of TMB solution at room temperature were added and the plate was shaken for around 5 minutes covering the 96-well plate with the aluminum foil (the solutions turn to blue).

50 μL of H2SO4 2 M were added in each well to stop the reaction (the solutions turn to yellow).

The 96-well plate was read at 450 nm.

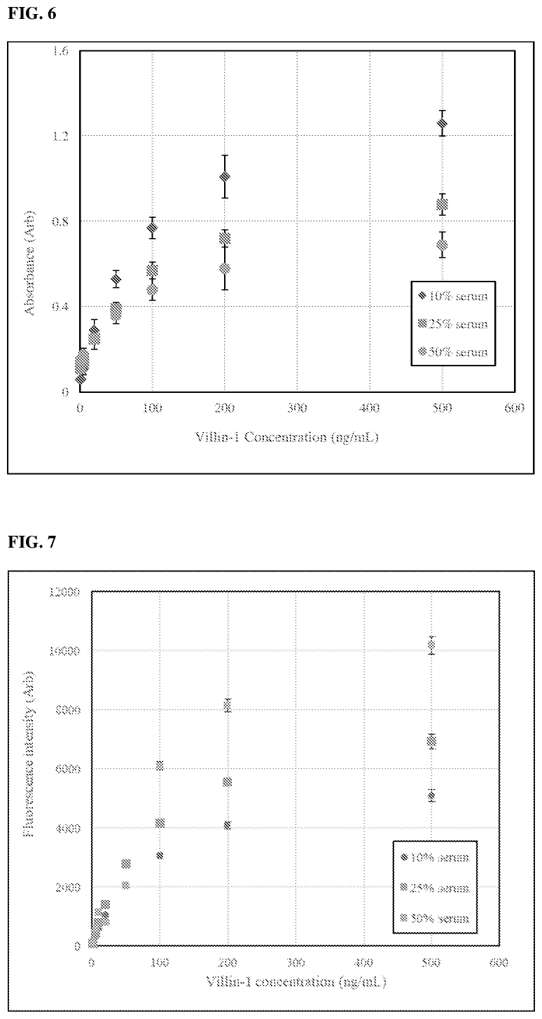

FIG. 6 shows the absorbance values for the sandwich ELISA performed on different concentrations of Villin-1 (0.005-100 ng/ml) in 10, 25 and 50% of human serum with the polyclonal R814 (Ab1) and N-terminal-3E5G11 (Ab2) at 5 μg/ml. Controls in 50% serum without Villin-1 were also analyzed. The absorbance measurements show that Villin-1 was detected in the different concentrations of serum until 2.0 ng/ml. Below this concentration the absorbance signal is low (ca. 0.05) and did not change by increasing the percentage of serum. A higher background (ca. 0.1) was observed from the control where both anti-Villin-1 antibodies (Ab1 and Ab2) were used with 50% of serum solution.

### EXAMPLE 4: SPM BEAD ASSAY FOR VILLIN-1 IN HUMAN SERUM

Based on the results presented in Example 3 (Villin-1 ELISA performed by adsorbing the polyclonal antibody onto the plate) another assay was carried out to detect the Villin-1 in serum using streptavidin-PEG functionalized 1 μm beads with the biotinylated polyclonal antibody.

### New Reagents

Biotinylation of Villin-1 antibody—The Villin-1 rabbit antibodies were biotinylated with Biotin-XX Microscale Protein Labeling Kit (Thermo Fisher Scientific, USA). In a 1.5 mL low-binding Eppendorf tube, a solution of R814 and 3E5G11 antibodies ca. 0.9 g/L in PBS with 10% of NaHCO₃ sol. 1 M pH 8.3 was reacted with Biotin-XX-NHS 4.98 μmol/μL solution in water at 12-fold molar ratio. The antibody solution was shaken for 1 hour at room temperature and then washed on a 0.5 mL Zeba™ spin desalting column with 7 kDa MWCO (Thermo Fisher Scientific Inc., USA). The antibody was stored at −20° C. in PBS buffer with 10% glycerol for further use.

Anti-Villin-1 beads were prepared by reaction of biotinylated Villin-1 antibody with streptavidin beads with polyethylene glycol brush monolayers. These beads were washed three times with PBST 0.05% buffer and resuspended in PBST to a concentration of 1.4 g/L in a 1.5 mL Eppendorf® LoBind tubes. The biotinylated antibody was added to the tube to a concentration of 25 μg/mL. The reaction was placed on a rotating wheel for 17 h at 4° C. before being collected using a magnet. The functionalized beads were washed two times by magnetic separation with PBST and suspended in 2% BSA in PBST to yield a 5 mg/mL solution of antibody functionalized beads.

The coverage of the antibodies on the beads was characterized with a secondary fluorescent antibody. For example, the R814 functionalized SPM beads were prepared with 1 mg/mL of R814-SPM beads in PBST were reacted with the Alexa 488 conjugated anti-IgG antibody (Thermo Fisher Scientific, USA) at 5 μg/mL in PBS in a 0.5 mL Eppendorf® LoBind tube. The tube was placed on a rotating wheel for 5 h at room temperature. The beads were then collected with a magnet, washed twice with PBST and then resuspended in PBST to a final concentration of 5 g/L. The fluorescence of the beads was then measured with a flow cytometer by dispersing 2 μL of beads solution in 300 μL of PBST. To ensure an optimal coverage of the beads surface with the biotinylated R814 antibody, the beads were reacted with increasing concentrations (8-38 μg/mL) of the antibody until the highest fluorescent signal was observed for a concentration of 25-28 μg/mL of the Villin-1 antibody.

Alexa Fluor™ 488 NHS Ester (Succinimidyl Ester) functionalization of anti-Villin antibody—The Villin-1 mouse antibody 3E5G11 was labeled with Alexa fluor 488 kit (Thermo Fisher Scientific, USA). In a 1.5 mL low-binding Eppendorf tube, a solution of 3E5G11 antibody ca. 0.9 g/L in PBS with 10% of NaHCO₃ sol. 1 M pH 8.3 was reacted with Alexa fluor 488-XX-NHS 4.98 μmol/μL solution in water at 12-fold molar ratio. The antibody solution was shaken for 1 hour at room temperature and then washed on a 0.5 mL Zeba™ spin desalting column with 7 kDa MWCO (Thermo Fisher Scientific Inc., USA). The antibody was stored at −20° C. in PBS buffer with 10% glycerol for further use.

### Procedure

Dilutions of human serum were prepared in 50% PBST.

50 μL of each Villin-1 dilution was placed in two 0.5 mL Eppendorf® LoBind tubes.

Villin-1 antibody functionalized beads were added to each tube to a final concentration of 0.5 mg/mL and let to react for 15 min on a shaker at 37° C.

After the required reaction time, the beads were collected by placing the tubes next to a NdBFe magnet with a field strength of 2.5 kGauss until the majority of the beads was on the side of the tube and the solution was clear.

The supernatant was removed, and the beads washed 1× with PBST by magnetic separation.

The SPM beads were reacted for another 15 min followed by the addition of Alexa-488 conjugate (this step may be removed for serum samples if polyethylene glycol brush coated beads are used).

After another the incubation step the SPM beads solution was directly analyzed by flow cytometry (FC) where the beads fluorescence was measured. Beads fluorescence intensity was measured. The assay sample was placed into the flow cytometer instrument and the sample was analysed until 15,000 events had been recorded in the gated area of the scatter plots with the fluorescence channel 1 area (FL1-Area) against the foreword scattering height (FSC-Height) of the beads (threshold in 80,000). Each assay was run in triplicate and the average values are plotted in all graphs.

Both combinations of the two Villin-1 antibodies, i.e., 3E5G11 and R814, were tested. The fluorescent assay was performed with SPM beads functionalized with both antibodies in buffer and in serum and the performance compared. Similar sensitivity was observed for the assay when antibodies 3E5G11 or R814 were used as capture antibody Ab1 in buffer. However, the sensitivity of the assay performed with 3E5G11 antibody functionalized SPM beads drastically decreased in 50% serum. For the R814 antibody functionalized SPM beads, a decrease in sensitivity was observed moving from buffer to 50% serum. In this case, the limit of detection was calculated to be 1 ng/ml in buffer and 5 ng/ml in 10, 25 and 50% serum.

FIG. 7 shows a graph reporting the values for the sandwich fluorescence assays performed on different concentrations of Villin-1 in 10, 25 and 50% of human serum with 1 μm polyethylene oxide brush coated SPM beads with R814 (Ab1) and the N-terminal-3E5G11 (Ab2) antibody. Controls in 50% serum without Villin were also analyzed.

As shown in FIG. 7, Villin-1 was detected until 5 ng/ml in 50% serum solutions. This time the fluorescence background signal was also much lower than in the previous ELISA assays.

The Villin-1 SPM bead assay has also been performed in human serum with streptavidin functionalized 2.7 μm diameter DYNABEADS M-270 beads. The results of these assays had a poorer sensitivity and longer response time, which was attributed to the multiple wash steps that were required. It is understood to those skilled in the art that the Villin-1 SPM bead assay can also be executed with enzymatic (horse radish peroxidase or alkaline phosphatase) assays the produce changes in color. Other optical reporters can be used to detect the formation of the antibody sandwich, e.g., electrochemiluminescence assays, and are likely to result in higher sensitivities.

### EXAMPLE 5: SPM BEAD ASSAY FOR VILLIN-1 IN RAT MODELS (SPM BEAD ASSAY FOR VILLIN-1 IN RAT SERUM)

Based on the results presented in Example 4 another assay was carried out to detect the Villin-1 in serum using the streptavidin functionalized, polymer coated 1 micrometer diameter beads with the biotinylated R814 polyclonal antibody.

The Rat AMI model samples were prepared by inducing intestinal ischemia in the intestines of rats by restricting blood flow in their intestines for 60 minutes. The blood flow was then allowed to return for 60 minutes and blood samples were collected.

### Procedure

Dilutions of rat serum were prepared in 50% PBST.

50 μL of each sample was placed in two 0.5 mL Eppendorf® LoBind tubes.

R814 anti-Villin-1 antibody functionalized beads were added to each tube to a final concentration of 0.5 mg/mL and let to react for 15 min on a shaker at 37° C.

After the required reaction time, the beads were collected by placing the tubes next to a NdBFe magnet with a field strength of 2.5 kGauss until the majority of the beads were on the side of the tube and the solution was clear.

The supernatant was removed, and the beads washed 1× with PBST by magnetic separation.

The SPM beads were reacted for another 15 min followed by the addition of the N-terminal 3E5G11 Alexa-488 conjugate.

After another the incubation step the SPM beads solution was directly analyzed by FC where the beads fluorescence was measured. Table 1 shows Villin-1 superparamagnetic bead assay. Fluorescence values for the sandwich ELISA on superparamagnetic beads for the concentrations of Villin-1 performed on different animal model samples (Ischemia, sham, and control). The results of this assay demonstrate that AMI can be detected in 5 out of 6 samples.

The Villin-1 SPM bead assay has also been performed on the rat AMI model samples with streptavidin functionalized 2.7 μm diameter DYNABEAD M-270 beads. These assays had a poorer sensitivity and longer response time, which was attributed to the multiple wash steps that were required. It is understood to those skilled in the art that the Villin-1 SPM bead assay can also be executed with enzymatic (horse radish peroxidase or alkaline phosphatase) assays the produce changes in color. Other optical reporters can be used to detect the formation of the antibody sandwich, e.g., electrochemiluminescence assays, and are likely to result in higher sensitivities.

### EXAMPLE 6: LATERAL FLOW ASSAY FOR THE DETECTION OF VILLIN-1

This is an illustrative, non-limiting, example of a lateral flow assay (currently in optimization) that could be used to evaluate the levels of Villin-1 in samples to diagnose subjects with AMI as well as other pathological conditions of the intestine.

### Materials

The different material used are summarized in Table 2.

Summary of the material used in development of a lateral flow assay prototype to evaluate the levels of Villin-1 in samples.

| Material | Description |
|---|---|
| Absorbent pad | Cellulose fiber from Merck |
| Detection pad | Cellulose ester membrane (4 mm backed), with a |
| | nominal capillary flow time of 180 seconds |
| | per 4 cm from Merck |
| Nanoparticles | 30 nm gold nanoparticles from BBI |
| Capture antibody | Rabbit polyclonal R814 anti-Villin-1 antibody |
| | (epitope on the C-terminal part) from Cell |
| | Signaling Technology |
| Detection antibody | Mouse monoclonal 3E5G11 anti-Villin-1 antibody |
| | (epitope on the N-terminal part) from Invitrogen |
| Control antibody | Rabbit anti-mouse secondary antibody (conjugated |
| | with HRP from Invitrogen) |
| Sample pad buffer | PBS with 0.5% BSA and 0.05% Tween-20 |
| Conjugate pad | PBS with 5% sucrose, 1% BSA and 0.5% Tween-20 |
| buffer | |
| Villlin-1 | Human recombinant Villin-1 generated in HEK293T |
| | (92.5 kDa) from Origene |

### Assay Preparation and Operation

The current lateral flow assay is constituted of two pads (the detection pad and the absorbent pad) that is herein referred to as a “half-strip” assay.
1. The first step is to prepare the nanoparticles conjugated with the detection antibody by passive adsorption:
   Gold nanoparticles are diluted for OD˜0.45 in borate buffer (pH 7) in 2 mL Protein low-binding Eppendorf tubes
   Detection 3E5G11 antibody is added to the gold nanoparticles (final concentration of 2.5 μg/mL)
   The pH of the borate buffer and the antibody concentration used in these steps have been optimized using an aggregation assay
   Solution is incubated at room temperature for 90 minutes, with agitation (650 rpm)
   For blocking, 100 μL of 1 mg/mL BSA in Milli-Q water is added to the solution 1 Solution is incubated at room temperature for 30 minutes, with agitation (650 rpm)
Solution is then centrifuged and the supernatant is removed. The red pellet, containing the nanoparticles conjugated with the detection antibody, is reconstituted in conjugate pad buffer (for a final OD˜3) and the solution is stored in the fridge until desired
2. The second step is to cut the detection pads and the absorbent pads at the required size:
   Absorbent pads are cut for ˜3 mm of width and 2 cm of length
   Detection pads are cut for ˜3 mm of width and 3 cm of length
3. The third step is to immobilize the capture and control antibodies on the detection pad at specific position:
   0.2 μL of the capture antibody (at concentration of 1 mg/mL) is deposed on the detection pad using a pipet, at ˜1.6 cm from the bottom of the pad (at the test line)
   0.2 μL of the control antibody (at concentration of 0.2 mg/mL) is deposed on the detection pad using a pipet, at ˜2.2 cm from the bottom of the pad (at the control line)
   After deposition of the antibodies, the detection pad was left to dry by incubation at 38° C. for ˜60 minutes
4. The fourth step is to assemble the detection pads and the absorbent pads
   The detection pads are placed on pieces of tape
   The absorbent pads are then deposed on the tape, with ˜2 mm of overlap with the top of the detection pads
   Delicate pression is applied to ensure that the pads are attached to the tapes and that contact is present between the two pads. The extra tape is then cut off
   The half-strips lateral flow assay are now ready to use
5. The fifth step is to prepare samples to be tested. In this optimization procedure, no clinical samples are required. They are replaced by a serial dilution of a known amount of recombinant human Villin-1 in sample pad buffer or in serum.
   Serial dilution of recombinant human Villin-1 are prepared in sample pad buffer, for final concentrations of 2000 ng/mL, 1000 ng/ml, 200 ng/mL, 100 ng/ml, 20 ng/ml and 2 ng/ml
   17.5 μL of each of these dilutions is added to 17.5 μL of either sample pad buffer (test of the half strip assay in 100% buffer) or serum (test of the half strip assay in 50% serum) for final concentration of Villin-1 in samples of 1000 ng/mL, 500 ng/mL, 100 ng/mL, 50 ng/mL, 10 ng/ml and 1 ng/mL. Additionally, a negative control is added, containing 17.5 μL of sample pad buffer with either 17.5 μL of sample pad buffer or 17.5 μL of serum
6. The sixth step is to incubate the serial dilutions of Villin-1 with the nanoparticles conjugated with the detection antibody in order to allow the binding of Villin-1 to the detection antibody:
   35 μL of the serial dilutions of Villin-1 (in 100% sample pad buffer and in 50% serum) are mixed with 30 μL of the nanoparticles conjugated with the detection antibody
   Solutions are left to incubate at 37° C. for 30 minutes, with agitation (650 rpm)
7. The seventh step is to add the half-strips lateral flow assays to the solutions containing serial dilution of Villin-1 and nanoparticles conjugated with the detection antibody. After the complete migration of the solution on the half-stick, 10 μL of sample pad buffer was added (as a running buffer) to ensure complete migration of the nanoparticles conjugate to the absorbent pad (detection pad going from a pinkish to a white color).

### Results

During the migration of the sample through the detection pad, Villin-1 (bound with the detection antibodies conjugated with the nanoparticles) specifically binds to the capture antibodies on the test line, revealing a red dot that will be more intense as the levels of Villin-1 in samples are higher. After that, the solution crosses the control line, where the control antibodies bind to the detection antibodies conjugated with the nanoparticles, to form a red dot, indicative of the validity of the test.

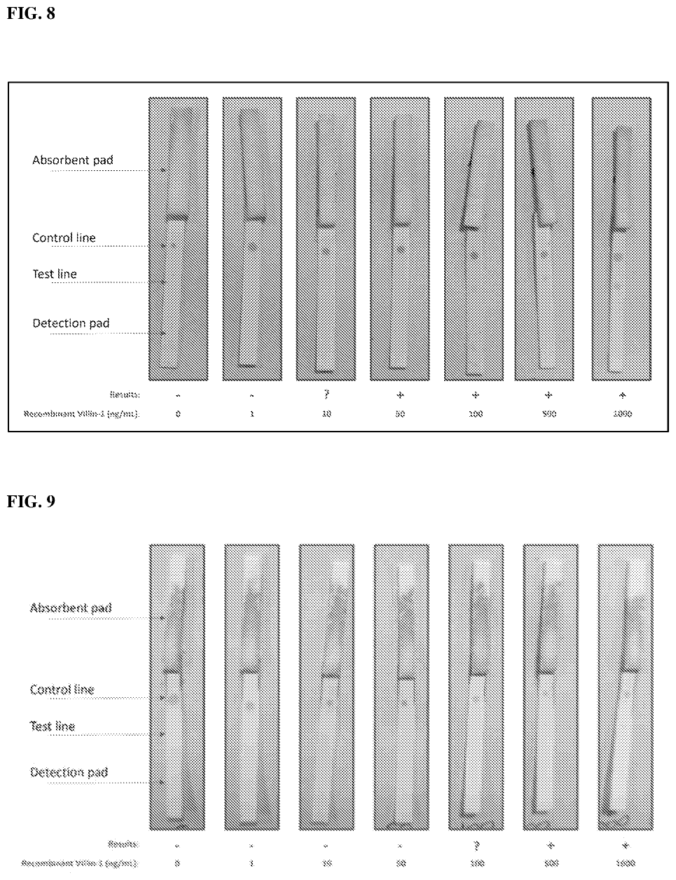

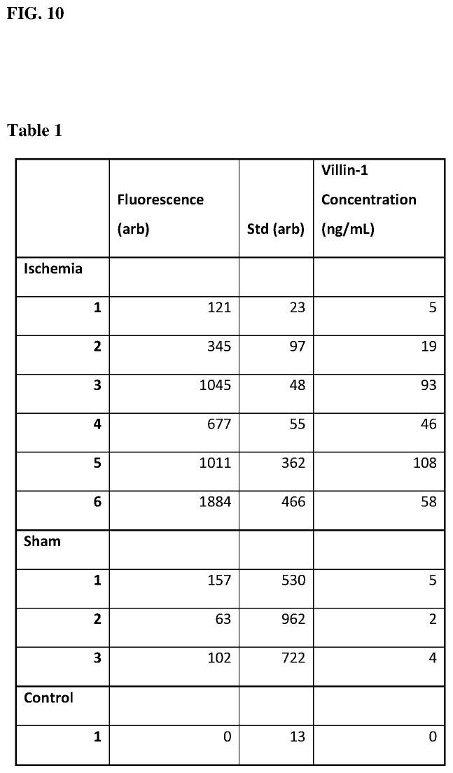

FIG. 8 represents a picture of the results of the half-strip lateral flow assays to analyze the levels of Villin-1 in buffer (100% sample pad buffer). A clear red dot is present at the control line in all half-strips, indicating that the results are valid. Naked eye detection of a red dot at the test line was possible for concentrations of Villin-1 of 50 ng/ml, 100 ng/ml, 500 ng/mL and 1000 ng/mL, with a color gradient representing the increased in the levels of Villin-1 between those samples. A very light signal was visible in the sample containing 10 ng/ml of Villin-1 but might not be strong enough for naked eye detection in all lighting conditions. It is possible that a specific reader could detect this low signal. Finally, no red dots were visible for the negative control (showing no unspecific binding of the nanoparticles to the test line) and for the sample containing 1 ng/mL of Villin-1 (showing that this level of Villin-1 is below the detection levels of the current prototype).

FIG. 9 represents a picture of the results of the half-strip lateral flow assays to analyze the levels of Villin-1 in 50% serum (50% serum, 50% sample pad buffer). A clear red dot is present at the control line in all half-strips, indicating that the results are valid. Naked eye detection of a red dot at the test line was possible for concentrations of Villin-1 of 500 ng/mL and 1000 ng/mL, with a color gradient representing the increased in the levels of Villin-1 between those samples. A very light signal was visible in the sample containing 100 ng/ml of Villin-1 but not strong enough for naked eye detection in all lighting conditions. It is possible that a specific reader could detect this low signal. Finally, no red dots were visible for the negative control (showing no unspecific binding of the nanoparticles to the test line) and for the sample containing 1 ng/mL, 10 ng/ml and 50 ng/mL of Villin-1 (showing that these levels of Villin-1 are below the detection levels of the current prototype).

### Discussion

The examples herein describe the manufacture and operation of a prototype of half-strip lateral flow assay that can be used to estimate the levels of Villin-1 in samples for diagnosis and prognosis of AMI and other pathological conditions of the intestine in subjects.

The limit of detection appears to be approximately 10 times higher when the serial dilution of Villin-1 are made in serum, as opposed to sample pad buffer. The likely explanation is that serum is a complex matrix composed of a multitude of compounds (including albumin, antibodies, antigens, hormones, enzymes, electrolytes and the like) that can affect the binding of the capture antibody and/or of the detection antibody to their target. To overcome this problem, despite further optimization of the current half-strip lateral flow assay, it would be possible to separate the Villin-1 from the other compounds found in serum (or in other complex samples such as plasma and blood) using filtration or other separation techniques.

Some of the steps that could help to improve the lateral flow assay performance include (1) test other antibody pairs that could offer better stability or quicker/stronger binding with their target, (2) use different type/concentration of buffers, stabilizers, blocking agents, detergents (etc.) to ensure proper flow of the samples and optimal conditions for the assay (for example for stabilizer: adding trehalose (˜1-10%) or alcohol (˜1-10%) to the conjugate pad buffer and/or to the capture/control antibodies could facilitate the preservation of the structure/function of the antibodies through drying cycles), (3) utilize a different conjugation method to attach the detection antibody on the nanoparticles (for example allowing the formation of a covalent bound to ensure that the detection antibody does not detach from the nanoparticle during the assay), (4) test different sizes of nanoparticles to find the optimal balance between high surface area and increased optical signal.

Moreover, the use of equipment for precise and reproducible construction of a lateral flow strip could also improve sensitivity and readability. For example, using a reagent dispenser system to strip the capture antibody and the control antibody on the detection pad would allow to have a thin test line and control line, at the exact same location, containing exactly the same amount of antibody, as opposed to the current manual pipetting of the antibodies, which is not reproducible. Similarly, the assembly of the pads using a specific laminated card would allow to precisely assemble the different pads together, instead of the current manual assembly. Likewise, the use of a programmable strip cutter to precisely and reproducibly cut the final strips would be a good alternative to replace the manual cutting of strips. All these change in equipment would allow for better precision and reproducibility of the lateral flow strips produced, whilst also ensuring a better flow of the sample through the strip and a better signal visualization.

For the creation of what would be referred to as a full-strip lateral flow assay, two pads will need to be incorporated in the present design of a half-strip lateral flow assay, the sample pad and the conjugate pad. To do that, a sample pad could be submerged in sample pad buffer before being dried for up to two hours in an oven at 38° C. A conjugate pad could be similarly submerged in conjugate pad buffer before being dried for up to two hours in an oven at 38° C. Afterwards, ˜30 μL of nanoparticles conjugated with detection antibody (produced as in Step 1 described above) could be added to the conjugate pad and further drying in the oven for up to two hours. After that, the conjugate pad would be added to the bottom of the half strip (produced as in Step 4 described above), on top of the detection pad, with ˜2 mm overlap. Similarly, the sample pad would be added on top of the bottom of the conjugate pad, with ˜2 mm overlap. After delicate pression is applied to ensure all pads are properly attached, the full lateral flow strip can be inserted in a housing cassette of the specific size and be stored in cool and dry conditions before use. When operated, the sample would be added to the hole of the cassette, to be deposited on the sample pad. The migration would then automatically start and results would be analyzed as described in the previous section. It is important to note that, in this format, the incubation time of the sample with the detection antibody conjugated with the nanoparticles will be lower than in the methodology explained earlier (for the half-strip assay), highlighting the importance of having antibodies with fast binding kinetics for Villin-1.

### REFERENCES

1. Sanson T G, O'Keefe K P. Evaluation of abdominal pain in the elderly. Emerg. Med. Clin. North Am. 1996; 14:615-627.
2. Kamin R A, Nowicki T A, Courtney D S, Powers R D. Pearls and pitfalls in the emergency department evaluation of abdominal pain. Emerg. Med. Clin. North Am. 2003; 21:61-72, vi.
3. Irvin T T. Abdominal pain: a surgical audit of 1190 emergency admissions. Br. J. Surg. 1989; 76:1121-1125.
4. Pitts S R, Niska R W, Xu J, Burt C W. National Hospital Ambulatory Medical Care Survey: 2006 emergency department summary. Natl. Health Stat. Report. 2008; 1-38.
5. Cordell W H, Keene K K, Giles B K, Jones J B, Jones J H, Brizendine E J. The high prevalence of pain in emergency medical care. Am. J. Emerg. Med. 2002; 20:165-169.
6. Lukens T W, Emerman C, Effron D. The natural history and clinical findings in undifferentiated abdominal pain. Ann. Emerg. Med. 1993; 22:690-696.
7. Lewis L M, Banet G A, Blanda M, Hustey F M, Meldon S W, Gerson L W. Etiology and clinical course of abdominal pain in senior patients: a prospective, multicenter study. J. Gerontol. A Biol. Sci. Med. Sci. 2005; 60:1071-1076.
8. Kizer K W, Vassar M J. Emergency department diagnosis of abdominal disorders in the elderly. Am. J. Emerg. Med. 1998; 16:357-362.
9. Bugliosi T F, Meloy T D, Vukov L F. Acute abdominal pain in the elderly. Ann. Emerg. Med. 1990; 19:1383-1386.
10. Fenyo G. Acute abdominal disease in the elderly: experience from two series in Stockholm. Am. J. Surg. 1982; 143:751-754.
11. Evennett N J, Petrov M S, Mittal A, Windsor J A. Systematic review and pooled estimates for the diagnostic accuracy of serological markers for intestinal ischemia. World J. Surg. 2009; 33:1374-1383.
12. Acosta S. Epidemiology of mesenteric vascular disease: clinical implications. Semin. Vasc. Surg. 2010; 23:4-8.
13. Kassahun W T, Schulz T, Richter O, Hauss J. Unchanged high mortality rates from acute occlusive intestinal ischemia: six year review. Langenbecks Arch. Surg. 2008; 393:163-171.
14. Schoots I G, Koffeman G I, Legemate D A, Levi M, van Gulik T M. Systematic review of survival after acute mesenteric ischemia according to disease aetiology. Br. J. Surg. 2004; 91:17-27.
15. Park W M, Gloviczki P, Cherry K J, Jr., Hallett J W, Jr., Bower T C, Panneton J M, et al. Contemporary management of acute mesenteric ischemia: Factors associated with survival. J. Vasc. Surg. 2002; 35:445-452.
16. Carver T W, Vora R S, Taneja A. Mesenteric Ischemia. Crit Care Clin. 2016; 32:155-171.
17. Corcos O, Nuzzo A. Gastro-intestinal vascular emergencies. Best. Pract. Res. Clin. Gastroenterol. 2013; 27:709-725.
18. Demir I E, Ceyhan G O, Friess H. Beyond lactate: is there a role for serum lactate measurement in diagnosing acute mesenteric ischemia? Dig. Surg. 2012; 29:226-235.
19. Clair D G, Beach J M. Mesenteric Ischemia. N. Engl. J. Med. 2016; 374:959-968.
20. Acosta S, Nilsson T. Current status on plasma biomarkers for acute mesenteric ischemia. J. Thromb. Thrombolysis. 2012; 33:355-361.
21. Zhao Y, Yin H, Yao C, Deng J, Wang M, Li Z, et al. Management of Acute Mesenteric Ischemia: A Critical Review and Treatment Algorithm. Vasc. Endovascular. Surg. 2016; 50:183-192.
22. Tilsed J V, Casamassima A, Kurihara H, Mariani D, Martinez I, Pereira J, et al. ESTES guidelines: acute mesenteric ischaemia. Eur. J. Trauma Emerg. Surg. 2016; 42:253-270.
23. Bretscher A, Weber K. Villin: the major microfilament-associated protein of the intestinal microvillus. Proc. Natl. Acad. Sci. U.S.A. 1979; 76:2321-2325.
24. Athman R, Louvard D, Robine S. The epithelial cell cytoskeleton and intracellular trafficking. III. How is Villin involved in the actin cytoskeleton dynamics in intestinal cells? Am. J. Physiol Gastrointest. Liver Physiol. 2002; 283: G496-G502.
25. Khurana S, George S P. Regulation of cell structure and function by actin-binding proteins: villin's perspective. FEBS Lett. 2008; 582:2128-2139.
26. Hinnebusch B F, Ma Q, Henderson J W, Siddique A, Archer S Y, Hodin R A. Enterocyte response to ischemia is dependent on differentiation state. J. Gastrointest. Surg. 2002; 6:403-409.
27. Bacchi C E, Gown A M. Distribution and pattern of expression of villin, a gastrointestinal-associated cytoskeletal protein, in human carcinomas: a study employing paraffin-embedded tissue. Lab Invest. 1991; 64:418-424.
28. Salbe C, de C P, Bonneton C, Manet S, Almeida A, Magdelenat H, et al. Illegitimate villin transcripts in normal bone marrow precludes detection of colon cancer micrometastases. Int. J. Biol. Markers. 2000; 15:41-43.
29. Arango D, Al-Obaidi S, Williams D S, Dopeso H, Mazzolini R, Corner G, et al. Villin expression is frequently lost in poorly differentiated colon cancer. Am. J. Pathol. 2012; 180:1509-1521.
30. Dudouet B, Robine S, Huet C, Sahuquillo-Merino C, Blair L, Coudrier E, et al. Changes in villin synthesis and subcellular distribution during intestinal differentiation of HT29-18 clones. J. Cell Biol. 1987; 105:359-369.
31. Ran, Y F, Fields, C, Muzard, J, Liauchuk, V, Carr, M, Hall, W and Lee, G U, Rapid, highly sensitive detection of herpes simplex virus-1 using multiple antigenic peptide-coated superparamagnetic beads, ANALYST (2014) DI 10.1039/c4an00774c

Other embodiments of the invention will be apparent to those skilled in the art from consideration of the specification and practice of the invention disclosed herein. It is intended that the specification and examples be considered as exemplary only.

What is claimed is:

1. A method of detecting and treating a pathological condition in an intestinal tract of a subject, such as a human, comprising:
contacting a biological sample from the subject with an analyte receptor and detecting binding between the analyte receptor and the analyte when the analyte is present in the biological sample; and
diagnosing the subject with the pathological condition when the analyte in the sample is detected and is present in an amount that exceeds a predetermined level, and
wherein the analyte has a receptor and is chosen from Villin-1, α-glutathione S-transferase, and intestinal-fatty acid binding protein (I-FABP).

2. The method of claim 1, wherein the pathological condition is selected from intestinal ischemia such as acute mesenteric ischemia, necrotizing enterocolitis, inflammatory bowel disease, and bowel graft rejection.

3. The method of claim 1, wherein the pathological condition is intestinal ischemia such as acute mesenteric ischemia, and optionally, after diagnosing the subject with intestinal ischemia such as acute mesenteric ischemia, performing a contrast angiography to confirm the diagnosis and/or to detect a blockage or defect in an affected blood vessel, such as an artery, vein, or capillary.

4. The method of claim 1, wherein the detecting binding between the analyte and the analyte receptor is chosen from:
a Villin-1 and an anti-Villin-1 antibody performed by an immunoassay, such as a lateral flow assay;
a α-glutathione S-transferase and an anti-α-glutathione S-transferase antibody performed by an immunoassay, such as a lateral flow assay; and
an intestinal-fatty acid binding protein (I-FABP) and an anti-I-FABP antibody performed by an immunoassay, such as a lateral flow assay.

5. The method of claim 1, after diagnosing the subject with the pathological condition, further comprising, administering an effective amount of an anticoagulant, antibiotic, or both, to the subject or performing surgery to treat the pathological condition.

6. The method of claim 5, wherein the subject has one or more symptoms chosen from abdominal pain; an urgent need to have a bowel movement; frequent, forceful bowel movements; abdominal tenderness or distention; blood in the subject's stool; and mental confusion.

7. The method of any one of claims 1-6, wherein the subject has abdominal pain.

8. The method of any one of claims 1-6, thereafter comprising initiating or changing treatment of intestinal ischemia such as acute mesenteric ischemia, comprising administering to the diagnosed subject an effective amount of one or more medicines for the treatment of intestinal ischemia such as acute mesenteric ischemia or performing surgery to treat the pathological condition, such as, acute mesenteric ischemia.

9. The method of claim 8, wherein the one or more medicines comprise one or more anticoagulants.

10. The method of claim 6, wherein the anticoagulants are chosen from warfarin, heparin, rivaroxaban (Xarelto) dabigatran (Pradaxa) apixaban (Eliquis), and edoxaban (Lixiana).

11. The method of claim 8, wherein the one or more medicines comprises antibiotics.

12. The method of claim 11, wherein the antibiotics are chosen from
Penicillins, such as penicillin V potassium, amoxicillin, amoxicillin/clavulanate (Augmentin);
Tetracyclines, such as doxycycline, tetracycline, and minocycline;
Cephalosporins, such as cefuroxime (Ceftin), ceftriaxone (Rocephin), and Cefdinir (Omnicef);
Quinolones, such as ciprofloxacin (Cipro), levofloxacin (Levaquin), and moxifloxacin (Avelox);
Lincomycins, such as clindamycin (Cleocin) and lincomycin (Lincocin);
Macrolides, such as azithromycin (Zithromax), clarithromycin (Biaxin), and erythromycin;
Sulfonamides, such as sulfamethoxazole-trimethoprim (Bactrim, Bactrim DS, Septra), sulfasalazine (Azulfidine), and sulfisoxazole (optionally combined with erythromycin);
Glycopeptides, such as dalbavancin (Dalvance), oritavancin (Orbactiv), telavancin (Vibativ), and vancomycin (Vancocin);
Aminoglycosides, such as gentamicin, tobramycin, and amikacin; and
Carbapenems, such as imipenem/cilastatin (Primaxin), meropenem (Merrem), doripenem (Doribax), and ertapenem (Inanz).

13. The method of claim 12, wherein the antibiotics are chosen from aminoglycosides, ampicillin, amoxicillin, amoxicillin/clavulanic acid (Augmentin), carbapenems (e.g., imipenem), piperacillin/tazobactam, quinolones (e.g., ciprofloxacin), tetracyclines, chloramphenicol, ticarcillin, trimethoprim/sulfamethoxazole (Bactrim), doxycycline, cephalexin, clindamycin, metronidazole, azithromycin, and levofloxacin.

14. The method of claim 12, wherein the antibiotics are chosen from broad spectrum antibiotics.

15. The method of any one of claims 1-6, wherein the condition is necrotizing enterocolitis.

16. The method of claim 15, wherein the subject has one or more symptoms chosen from abdominal distention (bloating or swelling); feedings stay in the stomach instead of moving through to the intestines as normal; bile-colored (greenish) fluid in the stomach; bloody bowel movements; and signs of infection such as apnea (stopping breathing), low heart rate, lethargy (sluggishness).

17. The method of claim 15 or 16, thereafter comprising initiating or changing treatment of necrotizing enterocolitis, comprising administering to the diagnosed subject an effective amount of one or more medicines for the treatment of necrotizing enterocolitis.

18. The method of claim 17, wherein the one or more medicines comprises antibiotics.

19. The method of claim 18, wherein the antibiotics are chosen from
Penicillins, such as penicillin V potassium, amoxicillin, amoxicillin/clavulanate (Augmentin);
Tetracyclines, such as doxycycline, tetracycline, and minocycline;
Cephalosporins, such as cefuroxime (Ceftin), ceftriaxone (Rocephin), and Cefdinir (Omnicef);
Quinolones, such as ciprofloxacin (Cipro), levofloxacin (Levaquin), and moxifloxacin (Avelox);
Lincomycins, such as clindamycin (Cleocin) and lincomycin (Lincocin);
Macrolides, such as azithromycin (Zithromax), clarithromycin (Biaxin), and erythromycin;
Sulfonamides, such as sulfamethoxazole-trimethoprim (Bactrim, Bactrim DS, Septra), sulfasalazine (Azulfidine), and sulfisoxazole (optionally combined with erythromycin);
Glycopeptides, such as dalbavancin (Dalvance), oritavancin (Orbactiv), telavancin (Vibativ), and vancomycin (Vancocin);
Aminoglycosides, such as gentamicin, tobramycin, and amikacin; and
Carbapenems, such as imipenem/cilastatin (Primaxin), meropenem (Merrem), doripenem (Doribax), and ertapenem (Inanz).

20. The method of claim 18, wherein the antibiotics are chosen from aminoglycosides, ampicillin, amoxicillin, amoxicillin/clavulanic acid (Augmentin), carbapenems (e.g., imipenem), piperacillin/tazobactam, quinolones (e.g., ciprofloxacin), tetracyclines, chloramphenicol, ticarcillin, trimethoprim/sulfamethoxazole (Bactrim), doxycycline, cephalexin, clindamycin, metronidazole, azithromycin, and levofloxacin.

21. The method of any one of claims 1-6, wherein the condition is inflammatory bowel disease, such as ulcerative colitis or Crohn's disease.

22. The method of claim 21, wherein the subject has one or more symptoms chosen from diarrhea, abdominal pain, fatigue and weight loss.

23. The method of any one of claims 21-22, thereafter comprising initiating or changing treatment of inflammatory bowel disease, comprising administering to the diagnosed subject an effective amount of the one or more medicines for the treatment of inflammatory bowel disease.

24. The method of claim 23, wherein the one or more medicines are chosen from antibiotics, anti-inflammatory drugs, and immune system suppressors.

25. The method of any one of claims 23-24, wherein the one or more medicines comprises antibiotics.

26. The method of any one of claims 24-25, wherein the antibiotics are chosen from Penicillins, such as penicillin V potassium, amoxicillin, amoxicillin/clavulanate (Augmentin);
Tetracyclines, such as doxycycline, tetracycline, and minocycline; Cephalosporins, such as cefuroxime (Ceftin), ceftriaxone (Rocephin), and Cefdinir (Omnicef);
Quinolones, such as ciprofloxacin (Cipro), levofloxacin (Levaquin), and moxifloxacin (Avelox);
Lincomycins, such as clindamycin (Cleocin) and lincomycin (Lincocin);
Macrolides, such as azithromycin (Zithromax), clarithromycin (Biaxin), and erythromycin;
Sulfonamides, such as sulfamethoxazole-trimethoprim (Bactrim, Bactrim DS, Septra), sulfasalazine (Azulfidine), and sulfisoxazole (optionally combined with erythromycin);
Glycopeptides, such as dalbavancin (Dalvance), oritavancin (Orbactiv), telavancin (Vibativ), and vancomycin (Vancocin);
Aminoglycosides, such as gentamicin, tobramycin, and amikacin; and
Carbapenems, such as imipenem/cilastatin (Primaxin), meropenem (Merrem), doripenem (Doribax), and ertapenem (Inanz).

27. The method of any one of claims 24-25, wherein the antibiotics are chosen from aminoglycosides, ampicillin, amoxicillin, amoxicillin/clavulanic acid (Augmentin), carbapenems (e.g., imipenem), piperacillin/tazobactam, quinolones (e.g., ciprofloxacin), tetracyclines, chloramphenicol, ticarcillin, trimethoprim/sulfamethoxazole (Bactrim), doxycycline, cephalexin, clindamycin, metronidazole, azithromycin, and levofloxacin.

28. The method of any one of claims 23-24, wherein the one or more medicines comprises anti-inflammatory drugs, such as corticosteroids and aminosalicylates.

29. The method of claim 28, wherein the anti-inflammatory drugs are chosen from mesalamine (such as Asacol HD, Delzicol, and others), balsalazide (Colazal) and olsalazine (Dipentum).

30. The method of any one of claims 23-24, wherein the one or more medicines comprises immune system suppressors, such as TNF-alpha inhibitors and biologics.

31. The method of claim 30, wherein the immune system suppressors are chosen from infliximab (Remicade), adalimumab (Humira), golimumab (Simponi), natalizumab (Tysabri), vedolizumab (Entyvio), and ustekinumab (Stelara).

32. The method of any one of claims 1-6, wherein the condition is bowel graft rejection.

33. The method of claim 32, wherein the subject has no symptoms or one or more symptoms chosen from fever, malaise, change in ostomy output (increased or decreased), intestinal bleeding, nausea, and vomiting.

34. The method of any one of claims 32-33, thereafter comprising changing treatment of bowel graft rejection, comprising administering to the diagnosed subject an effective amount of one or more medicines for the treatment of bowel graft rejection different than earlier treatments.

35. The method of claim 34, wherein the one or more medicines are chosen from antibiotics and anti-rejection medicines.

36. The method of any one of claims 34-35, wherein the one or more medicines comprises antibiotics.

37. The method of any one of claims 34-35, wherein the antibiotics are chosen from Penicillins, such as penicillin V potassium, amoxicillin, amoxicillin/clavulanate (Augmentin);
Tetracyclines, such as doxycycline, tetracycline, and minocycline;
Cephalosporins, such as cefuroxime (Ceftin), ceftriaxone (Rocephin), and Cefdinir (Omnicef);
Quinolones, such as ciprofloxacin (Cipro), levofloxacin (Levaquin), and moxifloxacin (Avelox);
Lincomycins, such as clindamycin (Cleocin) and lincomycin (Lincocin);
Macrolides, such as azithromycin (Zithromax), clarithromycin (Biaxin), and erythromycin;
Sulfonamides, such as sulfamethoxazole-trimethoprim (Bactrim, Bactrim DS, Septra), sulfasalazine (Azulfidine), and sulfisoxazole (optionally combined with erythromycin);
Glycopeptides, such as dalbavancin (Dalvance), oritavancin (Orbactiv), telavancin (Vibativ), and vancomycin (Vancocin);
Aminoglycosides, such as gentamicin, tobramycin, and amikacin; and
Carbapenems, such as imipenem/cilastatin (Primaxin), meropenem (Merrem), doripenem (Doribax), and ertapenem (Inanz).

38. The method of any one of claims 34-35, wherein the antibiotics are chosen from aminoglycosides, ampicillin, amoxicillin, amoxicillin/clavulanic acid (Augmentin), carbapenems (e.g., imipenem), piperacillin/tazobactam, quinolones (e.g., ciprofloxacin), tetracyclines, chloramphenicol, ticarcillin, trimethoprim/sulfamethoxazole (Bactrim), doxycycline, cephalexin, clindamycin, metronidazole, azithromycin, and levofloxacin.

39. The method of any one of claims 34-35, wherein the one or more medicines comprises anti-rejection medicines, such as immunosuppressive agents.

40. The method of claim 39, wherein the anti-rejection medicines are chosen from Tacrolimus (Prograf), Sirolimus (Rapamune), Steroids, and Everaloums.

41. The method of any one of the above claims, wherein the subject is chosen from humans (including infants) and other mammal animals, such as pets (dogs, cats, etc.), livestock (cattle, pigs, goats, sheep, horses, mules, donkeys, rabbits, and the like).

42. The method of any one of the above claims, wherein the biological sample is serum.

43. The method any one of the above claims, wherein the analyte receptor is an anti-Villin-1 antibody that is C-terminus or N-terminus type; an anti-α-glutathione S-transferase antibody that is C-terminus or N-terminus type; or an anti-intestinal-fatty acid binding protein (I-FABP) antibody C-terminus or N-terminus type.

44. The method any one of the above claims, wherein the analyte receptor is an anti-Villin-1 antibody that is polyclonal or monoclonal, an anti-α-glutathione S-transferase antibody that is polyclonal or monoclonal, or an anti-intestinal-fatty acid binding protein (I-FABP) antibody that is polyclonal or monoclonal.

45. The method any one of the above claims, wherein the analyte receptor is a Villin-1 receptor chosen from an anti-Villin-1 antibody, which is chosen from SP145 from, e.g., Invitrogen, UMAB230 from, e.g., OriGene, OTI3B3 from OriGene 3E5G11 from, e.g., Abcam, EPR3490 from, e.g., Abcam, VIL1 from, e.g., Abbexa, AS1A11 from, e.g., G Biosciences, VIL1/1314 from, e.g., enquire BioReagents, 1D2C3 from, e.g., Santa Cruz Biotechnology, OAGA00811 from, e.g., Aviva Systems Biology, OAEB02383 from, e.g., Aviva Systems Biology, and R814 from, e.g. Cell Signaling Technologies;
a α-glutathione S-transferase receptor chosen from an anti-α-glutathione S-transferase antibody, which is chosen from: e.g., anti-α-glutathione S-transferase antibody, Merck, e.g., GSTA1/α-glutathione S-transferase antibody, BioOrbyt, e.g., anti-α-glutathione S-transferase antibody, Sigma, e.g., anti-α-glutathione S-transferase antibody, Abbexa, e.g., Glutathione S Transferase alpha 1 (GSTA1) Rabbit Polyclonal Antibody, Origene, e.g., GSTA1 Polyclonal Antibody, ThermoFisher Scientific, e.g., Glutathione S-Transferase alpha 3, Antibodies-online.com, e.g., Glutathione S-Transferase alpha, Cloud-Clone Corp, e.g., Glutathione S-transferase alpha, Boster Bio, e.g., Glutathione S-transferase alpha, Bio-Techne, e.g., Glutathione S-transferase alpha, Absolute Antibody, or, e.g., Glutathione S-transferase alpha, Cambridge Bioscience; or
an intestinal-fatty acid binding protein (I-FABP) receptor chosen from an anti-I-FABP antibody, which is chosen from: e.g., Mouse anti-Human I-FABP/FABP2 Monoclonal Antibody (MBS246348), MyBioSource.com, e.g., Monoclonal Mouse anti-Human I-FABP/FABP2 Antibody, Lifespan Biosciences, e.g., anti-I-FABP antibody: Rabbit anti-Human I-FABP Polyclonal Antibody, MyBioSource.com, e.g., Mouse anti-Human FABP2 Monoclonal Antibody, ProteinTech, e.g., Fatty Acid Binding Protein 2, Intestinal (FABP2) Polyclonal Antibody, Biomatik, e.g., Recombinant Anti-I-FABP antibody, abCam, e.g., Rabbit Anti-Human FABP2/I-FABP pAb, Cell Sciences, e.g., Rat FABP2/I-FABP Biotinylated Antibody, R&D Systems, e.g., Rabbit Anti-Human FABP, Biorbyt, e.g., FABP2/I-FABP Antibody, Novus Biologicals, e.g., Intestinal Fatty Acid Binding Protein/I-FABP (FABP2) Antibody, Abbexa Ltd, e.g., Anti-FABP2 antibody, St. John's Laboratory, e.g., Anti-FABP2/I-FABP Antibody, BosterBio, e.g., Anti-FABP2, GeneTex, e.g., Anti-FABP2 Antibody, Rabbit Polyclonal, SionBiological, e.g., FABP2 antibody (Fatty Acid Binding Protein 2, Intestinal), Antibodies online, e.g., Rabbit Anti-FABP2, US Biological, e.g., FABP2 Polyclonal Antibody, Elabscience, e.g., I-FABP Polyclonal Antibody, G-Biosciences, e.g., I-FABP Antibody, Santa Cruz Biotechnology, Inc., e.g., I-FABP Antibody, Hycult Biotech, e.g., FABP2 Antibody, Thermo Fisher Scientific, e.g., FABP2 Antibody, NSJ Bioreagents, e.g., FABP2 Antibody, RayBiotech, e.g., FABP2 Antibody, AssayPro, e.g., FABP2 Antibody, Affinity Biosciences, e.g., FABP2 Antibody, OriGene Technologies, e.g., FABP2 Antibody, Cayman Chemical, e.g., FABP2 Antibody, Proteintech Group Inc, or e.g., FABP2 Antibody, ProSci.

46. The method of any one of the above claims, wherein detecting binding is between Villin-1 and the antibody;
α-glutathione S-transferase and the antibody; or
intestinal-fatty acid binding protein (I-FABP) and the antibody,
and wherein detecting binding lasts a period of time less than 120 minutes or 60 minutes or 30 minutes.

47. The method of any of the above claims, wherein detecting binding between Villin-1 and the antibody is performed by an immunological assay, such as a lateral flow assay; or detecting binding between α-glutathione S-transferase and the antibody is performed by an immunological assay, such as a lateral flow assay; or detecting binding between intestinal-fatty acid binding protein (I-FABP) and the antibody is performed by an immunological assay, such as a lateral flow assay.

48. The method of claim 47, wherein the immunological assay is chosen from ELISA.

49. The method of claims 47-48, wherein detecting binding between Villin-1 and the antibody is performed using a superparamagnetic bead comprising an anti-Villin-1 antibody; or detecting binding between α-glutathione S-transferase and the antibody is performed using a superparamagnetic bead comprising an anti-α-glutathione S-transferase antibody; or detecting binding between intestinal-fatty acid binding protein (I-FABP) and the antibody is performed using a superparamagnetic bead comprising an anti-I-FABP antibody.

50. The method of any of the above claims, wherein the predetermined level is a normal amount of analyte in a healthy control sample or a measured amount of analyte from a biological sample taken from the same subject at an earlier time or a level from defined severity to the intestinal tissue.

51. Any method of detecting a pathological condition in a subject's intestinal tract chosen from intestinal ischemia such as acute mesenteric ischemia, necrotizing enterocolitis, inflammatory bowel disease, and bowel graft rejection in a subject described herein.

52. A superparamagnetic bead comprising an anti-Villin-1 antibody; an anti-α-glutathione S-transferase antibody, or an anti-intestinal-fatty acid binding protein (I-FABP) antibody.

53. A superparamagnetic bead comprising a surface coating that binds Villin-1, α-glutathione S-transferase, or intestinal-fatty acid binding protein (I-FABP).

54. The superparamagnetic bead of claim 53, wherein said surface coating comprises a receptor or aptamer.

55. A kit comprising superparamagnetic bead comprising an anti-Villin-1 antibody, anti-α-glutathione S-transferase antibody, or anti-intestinal-fatty acid binding protein (I-FABP) antibody.
